(19) United States
(12) Patent Application Publication
(10) Pub. No.: US 2012/0231961 A1
(43) Pub. Date: Sep. 13, 2012

(54) METHODS AND SYSTEMS FOR SPORES DETECTION

(76) Inventors: Myron T. LA DUC, ARCADIA, CA (US); Kasthuri J. VENKATESWARAN, ARCADIA, CA (US); Bidyut MOHAPATRA, MOBILE, AL (US)

(21) Appl. No.: 13/417,238

(22) Filed: Mar. 10, 2012

Related U.S. Application Data
(60) Provisional application No. 61/451,251, filed on Mar. 10, 2011.

(51) Int. Cl. C12Q 1/68; G01N 21/64; C40B 30/00

(52) U.S. Cl. 506/7; 435/6.15; 435/6.12; 435/6.11

(57) ABSTRACT

Provided herein are methods and systems for the detection of spores in a sample which comprise permeabilizing a protein-based spore coat with a protein degrading agent comprising a non-ionic detergent and in particular with a specific mixture of various protein degrading agents comprising a non-ionic detergent to allow contact of spore nucleic acids with fluorescent reagents suitable for detection.

### CROSS REFERENCE TO RELATED APPLICATIONS

The present application claims priority to U.S. Provisional Application No. 61/451,251 filed on Mar. 10, 2011 which is herein incorporated by reference in its entirety.

### STATEMENT OF GOVERNMENT GRANT

The invention described herein was made in the performance of work under a NASA contract, and is subject to the provisions of Public Law 96-517 (35 USC 202) in which the Contractor has elected to retain title.

### FIELD

The present disclosure relates to methods and systems for spores detection and in particular for detecting viable and non-viable bacterial spores in materials and environmental samples.

### BACKGROUND

Development of various methods for detecting spores has been performed in connection with several applications used in medical, pharmaceutical, agricultural, aquacultural, water treatment, space exploration, and food processing industries.

In particular, spores detection has been typically directed to evaluating contamination of environments and materials of interest by environmental and/or pathogenic microorganisms, such as bacteria or fungi.

However, the inherent structure of spores and in particular of bacterial endospore typically interferes and in particular can preclude certain desired interactions between spores molecules (e.g., nucleic acids) and detection reagents, therefore leaving the accurate and reliable detection of spores (and in particular reliable detection of viable versus non-viable spores) particularly challenging.

### SUMMARY

Provided herein are methods and systems that, in several embodiments, allow interactions between detection reagents and molecules within the spore core for the purpose of detecting spores in a sample. In particular, methods and systems detailed herein facilitate, in several embodiments, the specific interaction of suitable detection reagents and spore nucleic acids for the purpose of detecting viable, non-viable, and/or total spores in a sample.

According to a first aspect, a method and system for permeabilizing a spore comprising a spore coating having a protein component is described. The method comprises contacting the spore with a protein degrading agent comprising a non-ionic detergent, with such contact occurring for a given time and under conditions to allow interaction of the protein degrading agent with the spore coating, and a degradation of the protein component of the spore coating. In some embodiments, the protein degrading agents further comprises additional agents, such as denaturing agents, and in particular reducing agents and/or enzymes such as proteases. In some embodiments, the contacting is performed to obtain a partial degradation of the protein component and/or partial degradation of the spore coating. The system comprises a non-ionic detergent and at least one additional protein degrading agent (e.g., one or more enzymes, and/or reducing agents) for simultaneous combined or sequential use in the method herein described.

According to a second aspect, a method and a system for detecting in a sample a spore having a coating with a protein component is described. The method comprises contacting the sample with a protein degrading agent comprising a non-ionic detergent for a time and under conditions allowing permeabilization of the spore coating to reagents for detection of nucleic acid comprised in the bacterial spore core, thus resulting in a treated sample. The method further comprises detecting the spore nucleic acid in the treated sample to indicate spores presence in the sample. The system comprises a non-ionic detergent and reagents for detection of the nucleic acid for simultaneous combined or sequential use in the method herein described. In some embodiments, the system can further comprise at least one additional protein degrading agent.

According to a third aspect, a method and system for detecting a viable spore in a sample are described. The method comprises contacting the sample with an intercalating agent capable of binding a first form of nucleic acid comprised in spores whose coats are penetrable to the intercalating agent. The contacting is performed for a time and under conditions to allow intercalation of the intercalating agent in the first form of nucleic acid and covalent linkage of the intercalating agent with the first form of nucleic acid following intercalation. In the method, the contacting of the sample with an intercalating agent results in a first treated sample. The method further comprises contacting the first treated sample with a protein degrading agent comprising a non-ionic detergent for a time and under condition to allow permeabilization of the spore to reagents for detection of a second form of nucleic acid comprised in spores not permeable to the intercalating agent to obtain a second treated sample and detecting of the second form of the nucleic acid in the second treated sample to indicate viable spores in the sample. The system comprises at least two of a non-ionic detergent, an intercalating agent and reagents for detection of the nucleic acid for simultaneous combined or sequential use in the method herein described. In some embodiments, the system can further comprise at least one additional protein degrading agent.

According to a fourth aspect, a method and a system for a live/dead assay for spores in a sample are described. The method comprises contacting a first unit of the sample with an intercalating agent capable of binding a first form of nucleic acids comprised in spores permeable to the intercalating agent. In the method, the intercalating agent is capable of emitting an intercalating labeling signal and the contacting performed for a time and under condition to allow intercalation of the intercalating agent in the first form of nucleic acid and covalent linkage of the intercalating agent with the first form of nucleic acid following intercalation. The contacting of the first unit of the sample results in a treated first unit. The method further comprises detecting the first form of nucleic acid in the treated first unit by detecting the intercalating labeling signal to indicate non-viable spores. The method further comprise contacting a second unit of the sample with a protein degrading agent comprising a non-ionic detergent for a time and under condition to allow permeabilization of spores to reagents for detection of nucleic acid comprised in bacterial spores to obtain a second treated sample. The method further comprises detecting the nucleic acid comprised in spores in the second treated sample to indicate viable and non-viable spores in the sample. The system comprises at least two of a non-ionic detergent, an intercalating agent and reagents for detection of the nucleic acid for simultaneous combined or sequential use in the method herein described. In some embodiments, the system can further comprise at least one additional protein degrading agent.

According to a fifth aspect, a method for a live/dead assay for spores in a sample is described, the method comprises contacting a first unit of the sample with an intercalating agent capable of binding a first form of nucleic acids comprised in spores permeable to the intercalating agent. In the method, the contacting is performed for a time and under condition to allow intercalation of the intercalating agent in the first form of nucleic acid and covalent linkage of the intercalating agent with the first form of nucleic acid following intercalation. The contacting results in a treated first unit. The method further comprises contacting the treated first unit with a protein degrading agent comprising a non-ionic detergent for a time and under condition to allow permeabilization of spores to reagents for detection of a second form of nucleic acid comprised in spore not permeable to the intercalating agent to obtain a secondly treated first unit. The method further comprises detecting the second form of nucleic acid in the secondly treated first unit to indicate viable spores in the first unit. The method further comprises contacting a second unit of the sample with a protein degrading agent comprising a non-ionic detergent for a time and under condition to allow permeabilization of spores to reagents for detection of a nucleic acid comprised in spores permeable or not permeable to the intercalating agent to obtain a treated second unit and detecting the nucleic acid in the treated second unit to indicate viable and non-viable spores in the second unit. The system comprises at least two of a non-ionic detergent, an intercalating agent and reagents for detection of the nucleic acid for simultaneous combined or sequential use in the method herein described. In some embodiments, the system can further comprise at least one additional protein degrading agent.

The methods and systems herein described allow in several embodiments rapid and sensitive qualitative and/or quantitative detection of spores from a sample and in particular detection of bacterial spores and more particularly endospores.

The methods and systems herein described also allow in several embodiments qualitative and/or quantitative detection of viable and/or non-viable spores as well as viable/non viable spore proportions.

The methods and systems herein described can be used in connection with applications wherein detection of spores and in particular of viable/non-viable spore is desired, including but not limited to medical application, biological analysis and diagnostics including but not limited to clinical applications, food industry applications (e.g. validating food processing technologies, and/or sterility and quality of food), pharmaceutical and medical equipment industries applications, (e.g. directed to spores detection as a marker for sterility), biosensors for bacterial detection in medical clinics, and microbial detection systems employed by governmental agencies, (e.g. directed to validating sterilization processes of USPS postal products, equipment and facilities, water treatment systems, and various public health applications).

The details of one or more embodiments of the disclosure are set forth in the accompanying drawings and the description below. Other features, objects, and advantages will be apparent from the description and drawings, and from the claims

### BRIEF DESCRIPTION OF DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows a gray scale picture illustrating results of a FISH assay performed with EUB338 probe for detection at 1,000× magnification. Panel A: shows B. pumilus SAFR-032 live spores; Panel B: shows B. pumilus SAFR-032 heat-killed spores (original probe color is fluorescent green).

FIG. 2 shows a gray scale picture illustrating results of a PMA-FISH assay performed with EUB338 probe for detection at 1,000× magnification. Panel A: shows B. pumilus SAFR-032 live spores; Panel B: shows B. pumilus SAFR-032 heat-killed spores (original probe color is fluorescent green).

FIG. 3 shows a gray scale picture illustrating results of a FISH assay of PMMA-encapsulated B. pumilus SAFR-032 spores released via PolyGone-500 performed using EUB338 probe for detection at Magnification 1,000× (original probe color is fluorescent green).

FIG. 4 shows a gray scale picture illustrating results of a PMA-FISH assay of PMMA-encapsulated B. pumilus SAFR-032 spores released via PolyGone-500 performed using EUB338 probe. For detection at Magnification 1,000× (original probe color is fluorescent green).

FIG. 5 shows a gray scale picture illustrating results of an Alexa-FISH assay performed using Bacillus pumilus-specific BP15 probe for detection at 1000× magnification (original probe color is fluorescent green). Panel A: Non-treated control spores, Panel B: PMMA-encapsulated spores released via PolyGone-500, Panel C: PMMA-encapsulated spores released via acetone.

FIG. 6 shows a gray scale picture illustrating results of an Alexa-FISH performed with universal EUB338 probe at 1000× magnification (original probe color is fluorescent green). Panel A: Non-treated control spores, Panel B: PMMA-encapsulated spores released via PolyGone-500, Panel C: PMMA-encapsulated spores released via acetone.

FIG. 7 shows a schematic of an exemplary method of the disclosure. Starting with a sample comprising live and dead spores, the method comprises removing extraneous DNA form the sample (S1) followed by treatment with PMA and light (S2). A permeability treatment, capable of permeating live and dead spores (S3), then gives a sample comprising permeable live spores with accessible nucleic acids and permeable dead spores still having their nucleic acids intercalated and cross-linked by the PMA and thus remaining inaccessible. Numerous nucleic acids detection techniques (S4) can then be performed to detect live spores.

FIG. 8. Shows a schematic illustration of an exemplary intercalating agent. The intercalating agent can be a molecule comprising a nucleic acid intercalating moiety and a functional group capable of forming a covalent bond with a nucleic acid.

### DETAILED DESCRIPTION

Methods and systems are described herein that in several embodiments, allow permeabilization of spores in a sample.

The term “sample” as used herein indicates a limited quantity of something that is indicative of a larger quantity of that something, including but not limited to solids and/or fluids from a biological environment, specimen, cultures, tissues, commercial recombinant proteins, synthetic compounds or portions thereof. Exemplary samples in the sense of the current disclosure include an environment sample collected from water, soil, air or the outer space, samples collected from a surface of a facility, equipment or system, food or pharmaceutical preparation.

Accordingly, the term “permeabilize” as used herein means to render permeable a substance, substrate, membrane or other material. The term “permeable” or “penetrable” as used herein refers to the ability of a substance, substrate, membrane or other material to absorb or allow the passage of substances (e.g. nucleic acids, intercalating agents, detection agents, and/or probes). The term “permeable” or “penetrable” can be a relative term to indicate permeability to specific reagents (e.g. of a particular size). Methods to measure the permeability of a material, before or after altering its permeability, will be apparent to a skilled person and can include techniques such as microscopy.

The term “spore” as used herein indicates a reproductive structure that is adapted for dispersal and surviving for extended periods of time in unfavorable conditions. Exemplary spores detectable with methods and systems herein described comprise spores from many bacteria, plants, algae, fungi and some protozoa. In some embodiments, detectable spores can be endospores. In general, spores comprise a protective protein-based coating which can comprise various spore proteins, forming a protein component of the spore coating. The protein component of a spore coating is typically comprised in more than one coating layers of the spore coating.

In some embodiments, the spores are bacterial spores. The term “bacteria” as used herein refers to single-cell prokaryotic microorganism species typically of a few micrometers in length and a wide range of shapes, including but not limited to Gram-negative bacteria and Gram-positive bacteria. The term “Gram-negative bacteria” refers to bacterial species that do not retain crystal violet dye in the Gram staining protocol. In contrast, the wording “Gram-positive bacteria” refers to bacterial species that are stained dark blue or violet by Gram staining. Several Gram-positive bacteria form endospores, including but not limited to the genus Bacillus and Clostridium. Bacillus bacteria are rod-shaped, aerobic or facultative, endospore-forming bacteria. The spores of Bacillus are particularly hard to lyse by either physical or chemical means due to its structure and composition. A spore core is surrounded by the core wall, a cortex and a spore coat.

The term “bacterial endospore” as used herein indicates a dormant and temporarily non-reproductive structure produced by certain bacteria, the formation of which is usually triggered under an unfavorable condition for bacteria, such as a lack of nutrients. The endospore typically consists of the bacterium's DNA and part of the bacterium cytoplasm, surrounded by a very tough outer coating, known as the endospore coat. Generally, when the environment becomes more favorable, the endospore can germinate to the metabolically active state, known as the vegetative state. Examples of bacteria able to form endospores comprise bacteria of the genus Bacillus and Clostridium.

For example, bacterial spores (endospores) produced by the genera Bacillus and Clostridium are a dormant form of cells that can persist for a long time in harsh conditions without dividing and display resistance towards chemical disinfectants, UV- and γ-radiation, and extreme pH, temperature, pressure and dryness (Ref. 71). These dormant spores are capable of passively monitoring the surrounding environmental conditions, and germinating into physiologically active vegetative cells upon exposure to favorable situations (Ref. 29). Several species of spore-forming bacteria are reported as pathogenic to humans and terrestrial and aquatic life and can survive hospital disinfection procedures (Ref. 70).

In embodiments herein described, methods and system comprise permeabilizing the bacterial spores or other spores such that the spores are permeable to reagents for detection of a nucleic acid comprised in the bacterial spore and that permeabilization is performed to substantially maintain the preexisting structure of the spore coat.

Therefore, in those embodiments, permeabilization according to methods herein described is performed with reagents that allow passage of the desired molecules through the spore coat minimizing the modifications of the existing spore coating (herein also partial degradation or weakening of the spore coating). Suitable reagents for weakening the spore coating are reagents able to degrade the protein component of the spore coating which comprise a non-ionic detergent typically in combination with additional reagents such as protease, chelating agents and/or chaotropic agents. In particular, the non-ionic detergent can be used, possibly in combination with other protein degrading agents, to weaken a spore structure or portion thereof which has a protein component and primarily comprises lipid. A suitable combination of the non-ionic detergent with protease, chelating agents and/or chaotropic agents can be identified by a skilled person based on the present disclosure in view of the specific spore structure and related spore coat layers.

For example, bacterial endospores have a protein based coating comprising various layers, some of which comprise a protein component. In particular, an exemplary bacterial endospore comprises an outermost layer referred to as an “exosporium” comprising primarily proteins, lipids, and polysaccharides; a second layer below the “exosporium” referred to as a “coat” comprising various spore proteins and comprising primarily insoluble proteins; a third layer below the “coat” referred to as a “cortex” comprising loosely linked peptidoglycans; a fourth layer below the “cortex” referred to as an “inner membrane” comprises primarily lipids; a “core” comprising nucleic acids of the spore. The inner membrane protects the spore “core” by providing impenetrable barrier to most compounds. Therefore, in order to access the nucleic acids of a spore such that various molecular detection techniques can be utilized, the layers of spore surrounding the spore's “core” can be permeabilized to access the nucleic acids and the spore coating is weakened.

Thus, in embodiments herein described permeabilization of a bacterial endospore can be performed according to a process in which permeabilizing of “exosporium” layer, the “coat” layer, the “cortex” layer, and the “inner membrane” layer is performed such that nucleic acids in the “core” of the bacterial endospore become accessible to agents capable of detecting the nucleic acids minimizing the modification of the existing spore structure and therefore allowing detection of the spore structure that provides a reliable indicator of the state and viability of the cell. In those embodiments, the non-ionic detergent can be used in particular reference to the permeabilization of the inner layer to minimize the modification of the inner layer.

In particular, some embodiments, a permeabilization of a viable bacterial endospore comprise four steps each directed at permeabilize an endospore layer. In particular, a first step comprises a permeabilization of the exosporium layer with one or more protein degrading agents suitable for permeabilizing this exosporium layer; a second step comprises a permeabilization of the coat layer with one or more protein degrading agents suitable for permeabilizing the coat layer; a third step comprises a permeabilization of the cortex layer with one or more protein degrading agents suitable for permeabilizing the cortex layer; and a fourth step comprises a permeabilization of the inner membrane layer with one or more protein degrading agents suitable for permeabilizing the inner membrane layer.

In some embodiments, protein degrading agents suitable for permeabilizing the exosporium layer comprise one or more detergents suitable to denature non-covalent bonds of proteins in combination with a protease to cleave peptidic bonds of proteins. In some embodiments the protease is proteinase-K. Proteinase K is an endopeptidase whose activity is not affected by of other denaturants, such as SDS, urea, and chelating agents. Thus Proteinase K is able to maintain its activity and synergistic function in the presence of other agents used during permeabilization and in particular, denaturants. In some embodiments the detergent is sodium dodecyl sulfate (SDS) or other anionic detergents/surfactants. Other exemplary detergents/surfactants include but are not limited to potassium lauryl sulfate, ammonium lauryl sulfate, and other anionic surfactants/detergents identifiable by one skilled in the art upon reading the present disclosure.

In some embodiments, protein degrading agents suitable for permeabilizing the coat layer comprise agents suitable to denature non-covalent interactions and/or intramolecular disulfide bonds of spore proteins. The agents comprise one or more detergents which can disrupt non-covalent interactions one or more metal chelators (e.g. EDTA, EGTA, 1,10-phenanthroline) which can remove metals from metal-bond proteins, one or more reducing agents (e.g. dithiothreitol (DTT), 2-mercaptoethanol) to reduce any disulfide bonds, and other suitable chaotropic agents (e.g. urea, guanidine chloride) capable of weakening hydrophobic interactions, and/or buffers (e.g. Tris). The term “chaotropic agent” as used herein refers to a substance which disrupts the structure of, and denatures, macromolecules such as, for example, proteins and nucleic acids (e.g. DNA and RNA). This disruption can be caused by, for example, the agent interfering with non-covalent intramolecular forces (e.g. hydrogen bonding and the hydrophobic effect), shielding charges and preventing formation of salt bridges, and by other mechanisms identifiable to a skilled person. Exemplary chaotropic agents include, but are not limited to, butanol, Ethanol, guanidinium chloride, lithium perchlorate, magnesium chloride, phenol, propanol, sodium dodecyl sulfate, thiourea, urea, and other agents identifiable to a skilled person. In some embodiments the one or more detergents suitable for permeabilizing the coat layer comprise nonionic and/or anionic detergents. In particular in some embodiments, the one or more detergents comprise SDS and Triton X-100, TWEEN (polysorbate) 60, Tween 20, potassium lauryl sulfate, ammonium lauryl sulfate, and other suitable nonionic and/or anionic detergents identifiable by one skilled in the art.

In some embodiments, agents suitable for permeabilize the cortex layer comprise one or more detergents agents suitable for degrading peptidoglycans, for example a glycosidase. In some embodiments, the agents suitable for removing or partially removing the cortex layer comprise lysozyme and mutanolysin. Other exemplary peptidoglycan degrading enzymes of the disclosure include but are not limited to muraminidase, peptidase and amidase.

In embodiments wherein weakening of the spore coating is desired, protein degrading agents suitable for permeabilizing the inner layer comprise a non-ionic detergent, possibly together with additional reagents suitable to solubilize lipids and denature proteins.

A suitable non-ionic detergent and possible related combination with solubilizing and or denaturing agent can be selected based on an extent to which the detergent is capable of permeabilizing a spore layer. Accordingly, a desired extent of permeabilization can be determined by methods identifiable by a skilled person, including, for example scanning electron microscopy (SEM) to analyze the surface of the spore after contacting the spore with the detergent. Other microscopy methods can also be used and are identifiable by a skilled person upon reading the present disclosure. Determination of a desired combination with solubilizing agent or denaturing agent can be performed following determination of permeability on a known spore coating.

The term “solubilize” as used herein with reference to solubilizing lipids, refers to a transfer of lipids comprised within a spore layer to a solvent (e.g. an aqueous solvent) in which the spores are dispersed. Solubilizing of lipids weaken the spore layers and can aid in compromising the integrity of the spore layers, thus permeabilizing the spore.

The term “denature” as used herein is defined to mean a process by which proteins and/or nucleic acids lose their native quaternary, tertiary, and/or secondary structure. Denaturation of a quaternary structure can comprise dissociation of protein sub-units and/or disruption of spatial arrangement of protein subunits. Denaturation of a tertiary structure can comprise disruption of covalent interactions between amino acid side chains such as disulfide bonds, disruption of non-covalent interactions such as dipole-dipole interactions between polar amino acid side chains and a surrounding solvent, and disruption of Van der Waals (e.g. induced dipole) interactions between non-polar amino acid side chains. Denaturation of a secondary structure can comprise a disruption of repeating patterns such as alpha-helices and beta-sheets to adopt, for example, random coil configurations. Denaturation of proteins comprised with spore layers can aid in compromising the integrity of the spore layers, thus permeabilizing the spore.

Thus a denaturing agent can be selected based on an extent to which the denaturing agent is capable of permeabilizing a spore layer in. Accordingly, a desired extent of permeabilization can be determined by methods identifiable by a skilled person, including, for example scanning electron microscopy (SEM) to analyze the surface of the spore after contacting the spore with the denaturing agent and additional methods identifiable by a skilled person.

In some embodiments, the agents suitable to solubilize lipids and denature residual proteins comprise one or more nonionic and/or anionic detergents and a metal chelator. In particular in some embodiments, the one or more detergents comprise SDS and Triton X-100 the metal chelator comprises EDTA or 1,10-phenanthoroline.

In some embodiments, protein degrading agents suitable to permeabilize an endospore coat show a synergistic effect. For example, in some embodiments, a combination of proteinase-K, lysozyme, mutanolysin, and Triton X-100 was shown to have a synergistic effect (See for example, Example 19).

In particular, non-ionic detergents can be suitable for permeabilization as they can provide permeabilization of a spore while maintaining a structural integrity of the spore (e.g. to prevent loss of nucleic acids in the core) which, while anionic detergents, under some conditions, and particularly with resistant spores such as B. pumilus SAFR-032 spores (See for example, Example 1), can compromise the structural integrity of the spores.

In some embodiments, efficiency of a permeabilization of endospores in a sample can be tested by performing FISH and visualizing the sample, for example, by microscopy. Microscopy can show independent spore subpopulation which can be visualized and thus their permeability confirmed. Small samples can also be removed from a total volume of the sample and tested concurrently. In particular, in some embodiments, a preliminary testing of efficiency of permeabilization allows selection of the proper detergent or combination of detergent. In particular one or more detergent can be contacted with a known sample of spores and the permeability of the spores to the detergent verified according to methods herein described and otherwise identifiable by a skilled person.

The term “detergent” as used herein refers to an amphiphilic (partly hydrophilic/polar and partly hydrophobic/non-polar) surfactant or a mixture of amphiphilic surfactants. Detergents can be broadly categorized according to the charge of their polar portion as “anionic” (negative charge; examples including, but not limited to alkylbenzenesulfonates and bile acids, such as deoxycholic acid), “cationic” (positive charge; examples including, but not limited to, quaternary ammonium and pyridinium-based detergents), “nonionic” (no charge; examples including, but not limited to, polyoxyethylene/PEG-based detergents such as Tween and Triton, and glycoside-based detergents such as HEGA and MEGA), and “zwitterionic” (no charge due to equal numbers of positive and negative charges on the detergent molecules; examples including, but not limited to, CHAPS and amidosulfobetaine-type detergents).

The term “glycoprotein” as used herein refers to proteins that contain oligosaccharide chains (glycans) covalently attached to polypeptide side-chains. Exemplary glycoproteins include, but are not limited to, follicle-stimulating hormone, luteinizing hormone, thyroid-stimulating hormone, human chorionic gonadotropin, alpha-fetoprotein, erythropoietin, collagens, mucins, transferrin, ceruloplasmin, immunoglobins, histocompatibility antigens, lectins, selectins, antibodies, calnexin, calreticulin, antibodies, and miraculin.

The term “metal chelator” as used herein refers to a molecule capable of binding or complexation of a bi- or multidentate ligand with a single metal ion. Examples of metal chelators include, but are not limited to, ethylenediaminetetraacetic acid (EDTA) and related salts, ethylenediamine, diethylenetriame, crown ethers, cryptands, cyclens, porphyrins, 1,10-phenanthroline, and catechol

In some embodiments a method for permeabilizing bacterial endospores according to embodiments of the disclosure can be used in connection with a number of nucleic acid detection techniques to detect nucleic acids bacterial endospores.

The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as ‘quantitation’), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “nucleic acid” as used herein indicates a polymeric form of nucleotides of any length, either ribonucleotides or deoxyribonucleotides, that comprise purine and pyrimidine bases, or other natural, chemically or biochemically modified, non-natural, or derivatized nucleotide bases. Nucleic acids of the embodiments of the current disclosure include Deoxyribonucleic acid (DNA), ribonucleic acid (RNA), or DNA copies of RNA (complementary DNA or cDNA), which may be isolated from natural sources, recombinantly produced, or artificially synthesized. The nucleic acids may exist as single-stranded or double-stranded and any chemical modifications thereof, provided only that the modification does not interfere with amplification of selected nucleic acids. For example, the backbone of the nucleic acid can comprise sugars and phosphate groups or modified or substituted sugar or phosphate groups, and a nucleic acid may comprise modified nucleotides, such as methylated nucleotides and nucleotide analogs.

In some embodiments, the method for permeabilizing bacterial endospores for subsequent detection techniques to detect nucleic acids in bacterial endospores comprises permeabilizing the bacterial endospores such that the spores are permeable to reagents for detection of a nucleic acid comprised in the bacterial spores. In those embodiments, the permeabilizing is followed by detecting the nucleic acid in the treated sample wherein the detected nucleic acid serve as an indicator of state and viability of bacterial spores in the sample.

In some embodiments, the nucleic acid detection methods can include fluorescence in situ hybridization (FISH), and in particular, Alexa-FISH.

The term “FISH”, or “fluorescence in situ hybridization”, as used herein refers to a cytogenetic technique that is used to detect and localize the presence or absence of specific DNA sequences on chromosomes. FISH uses fluorescent probes that bind to only those parts of the chromosome with which they show a high degree of sequence complementarity. Fluorescence microscopy can be used to find out where the fluorescent probe bound to the chromosomes. FISH can be used for finding specific features in DNA for use in genetic counseling, medicine, and species identification. FISH can also be used to detect and localize specific mRNAs within tissue samples. In this context, it can be used in some instances define the spatial-temporal patterns of gene expression within cells and tissues.

For example, permeabilized spores can be added to a solution of suitable FISH hybridization reagents and incubated for a suitable period of time to allow hybridization of labeled FISH probes to nucleic acids in the permeabilized spores. Following a stopping of the hybridization the spores can be filtered and excess reagents removed. Spores hybridized to labeled probes can then be detected, for example, by fluorescence microscopy.

In some embodiments, suitable nucleic acid detection methods can include DNA microarrays. The term “DNA microarray” as used refers to a collection of microscopic DNA spots attached to a solid surface. Forms of DNA microarrays include, but are not limited to, collections of orderly microscopic “spots”, called features, each with a specific probe attached to a solid surface, such as glass, plastic or silicon biochip and collections of microscopic polystyrene beads, each with a specific probe and a ratio of two or more dyes, which do not interfere with the fluorescent dyes used on the target sequence. Each DNA spot contains a specific DNA sequence, known as a probe. This can be a short section of a gene or other DNA element that are used to hybridize a cDNA or cRNA sample (called target) under high-stringency conditions. Probe-target hybridization is usually detected and quantified by detection of fluorophore-, silver-, or chemiluminescence-labeled targets to determine relative abundance of nucleic acid sequences in the target. Examples of uses of DNA microarrays include, but are not limited to, measuring the expression levels of large numbers of genes simultaneously, to genotype multiple regions of a genome gene expression profiling, comparative genomic hybridization, gene identification, chromatin immunoprecipitation on chip, DamID, SNP detection, and fusion transcript identification.

For example, permeabilized spores can be treated for a time and under conditions suitable to extract nucleic acid from the sample and then performing amplification of the extract nucleic acid. The extract can then be contacted with a DNA microarray comprising labeled probes for detection of nucleic acids extracted from the spores

In some embodiments, suitable nucleic acid detection methods can include terminal restriction fragment length polymorphism. The term “terminal restriction fragment length polymorphism” (TRFLP) as used herein refers to a technique for profiling of microbial communities based on the position of a restriction site closest to a fluorescently labeled end of an amplified gene. The method is based on digesting a mixture of PCR amplified variants of a single gene using one or more restriction enzymes and detecting the size of each of the individual resulting terminal fragments using a DNA sequencer. The result is a TRFLP profile where the X axis represents the sizes of the fragment and the Y axis represents their fluorescence intensity, and each peak corresponds to one genetic variant in the original sample while its height or area corresponds to its relative abundance in the specific community. Often, however, several different bacteria in a population might give a single peak on the TRFLP profile due to the presence of a restriction site for the particular restriction enzyme used in the experiment at the same position. To overcome this problem and to increase the resolving power of this technique a single sample can be digested in parallel by several enzymes (often three) resulting in three TRFLP profiles per sample each resolving some variants while missing others. Some ways in which data from TRFLP may be interpreted include, but are not limited to, a comparison of the general shapes of the TRFLP profiles of different samples, comparison of the TFRLP profile to a clone library, and comparison of the peaks in a TRFLP profile to a database to identify specific bacteria.

For example, permeabilized spores can be treated for a time and under condition suitable to extract nucleic acid from the sample followed by a performing contacting of the extract with labeled primers and other suitable reagents for TRFLP for a time and under conditions to allow hybridization of nucleic acids in the extract with the labeled primers and amplification of nucleic acids which can then be detected. In some embodiments, suitable nucleic acid detection methods can include denaturing gradient gel electrophoresis The term “denaturing gradient gel electrophoresis” (DGGE) as used herein refers to a form of electrophoresis which uses a chemical gradient to denature a sample of nucleic acids such as DNA and RNA (as well as occasionally proteins) as it moves across an acrylamide gel. In DGGE a small sample of DNA (or RNA) is applied to a electrophoresis gel that contains a denaturing agent capable of inducing DNA (or RNA) denaturation (“melting”). The DNA (or RNA) is subjected to increasingly extreme denaturing conditions and the melted strands fragment completely into single strands. Sequence differences in fragments of the same length often cause them to partially melt at different positions in the gradient and therefore “stop” at different positions in the gel. By comparing the melting behavior of the polymorphic DNA fragments side-by side on denaturing gradient gels, it is possible to detect fragments that have mutations in the first melting domain, and placing two samples side-by-side on the gel and allowing them to denature together, enables the detection of the smallest differences in two samples or fragments of DNA. Applications of DGGE include, but are not limited to, detection of mutations in mitochondrial DNA, detection of mutation of tumor protein 53 in pancreatic juices, and visualization of variations in microbial genetic diversity. For example, permeabilized spores can be treated for a time and under condition suitable to extract nucleic acid from the sample followed by a contacting of the extract with a gel and performing electrophoresis on the extract.

In some embodiments, suitable nucleic acid detection methods can include pyrosequencing. The term “pyrosequencing” as used herein refers to a method of DNA sequencing based on the “sequencing by synthesis” principle, which means taking a single strand of the DNA to be sequenced and then synthesizing its complementary strand enzymatically. A desired DNA sequence is able to be determined by light emitted upon incorporation of the next complementary nucleotide by the fact that only one out of four of the possible A/T/C/G nucleotides are added and available at a time so that only one letter can be incorporated on the single stranded template (which is the sequence to be determined). The intensity of the light determines if there is more than one of these “letters” in a row. The previous nucleotide letter is degraded before the next nucleotide letter is added for synthesis, allowing for the possible revealing of the next nucleotide(s) via the resulting intensity of light (if the nucleotide added was the next complimentary letter in the sequence). This process is repeated with each of the four letters until the DNA sequence of the single stranded template is determined. For example, permeabilized spores can be treated for a time and under condition suitable to extract nucleic acid from the sample followed by a contacting of the extract with pyrosequencing reagents and subsequent detection as would be understood by a skilled person.

In some embodiments, suitable nucleic acid detection methods can include reverse transcription polymerase chain reaction. The term “reverse transcription polymerase chain reaction” (RT-PCR) as used herein refers to a variant of the polymerase chain reaction (PCR). It is a laboratory technique commonly used in molecular biology where a RNA strand is reverse transcribed into its DNA complement (complementary DNA, or cDNA) using the enzyme reverse transcriptase, and the resulting cDNA is amplified using PCR. Reverse transcription PCR is not to be confused with real-time polymerase chain reaction (Q-PCR/qRT-PCR), which is also sometimes abbreviated as RT-PCR. Some uses of RT-PCR include use in the diagnosis of genetic diseases and, semiquantitatively, in the determination of the abundance of specific different RNA molecules within a cell or tissue as a measure of gene expression; use in the insertion of eukaryotic genes into prokaryotes; and use in studying the genomes of viruses whose genomes are composed of RNA, such as Influenza virus A and retroviruses like HIV. For example, permeabilized spores can be treated for a time and under condition suitable to extract nucleic acid from the sample. The extract can be contacted with RT-PCR reagents for a time and under conditions to allow amplification of the nucleic acids followed by a detection as would be understood by a skilled person

In some embodiments, suitable nucleic acid detection methods can include real-time PCR The term “real-time PCR”, “real-time quantitative PCR” or “qPCR” as used herein indicate a laboratory technique based on the polymerase chain reaction (PCR), which is used to amplify and simultaneously quantify a target DNA molecule. For one or more specific sequence in a DNA sample, real-time PCR enables both detection and quantification. The quantity can be either an absolute number of copies of the amplified DNA molecules or a relative amount when normalized to the DNA template input or additional normalizing genes or DNA molecule. The real-time PCR procedure follows the general principle of polymerase chain reaction with a key feature that the amplified DNA is detected as the reaction progresses i.e. in real time. Two common methods for detection of products of DNA amplification in real-time PCR are detecting non-specific fluorescent dyes that intercalate with any double-stranded DNA and detecting sequence-specific DNA probes consisting of oligonucleotides that are labeled with a fluorescent reporter which permits detection only after hybridization of the probe with its complementary DNA target. Real-time PCR can be combined with reverse transcription to quantify RNA molecule present in a sample. For example, permeabilized spores can be treated for a time and under condition suitable to extract nucleic acid from the sample. The extract can be contacted with qPCR reagents for a time and under conditions to allow amplification of the nucleic acids followed by a detection as would be understood by a skilled person.

In some embodiments, FISH is used to detect bacterial endospores. FISH microscopy can facilitate a simultaneous identification and enumeration of microorganisms without isolation and cultivation according to procedure identifiable by a skilled person. In the FISH method herein described, a fluorescently labeled oligonucleotide probes can seep through permeabilized viable bacterial endospores and hybridize to complementary rRNA sequences. This can allow for a screening of a particular subset of cell lineages present in a heterogeneous population of microorganisms or depending on a selected probe (e.g. specific or universal) can detect a general presence of spores. Typically, oligonucleotide probes used for FISH analysis are designed to target the 16S rRNA sequences of a particular taxonomic group of microbes, as these sequences are highly conserved compared to other protein-encoding genes according to procedures identifiable by a skilled person. Coupling of Alexa-fluor® dyes, known as Alexa-FISH, can provide a sensitive technique for spore detection.

In some embodiments, wherein Alexa-FISH is used to detect bacterial endospores according to embodiments herein described, less than 100 spores can be detected and in particular, as low as a single spore can be detected. Thus, embodiments of the disclosure can be suitable for diagnostic purposes.

FISH can be a particularly suitable method for detecting nucleic acids in the embodiments herein described as FISH can provide an ability to visualize and enumerate viable spores. Other detection methods, as previously mentioned (e.g. qPCR) can also be used to detect nucleic acids in embodiments of the disclosure.

In embodiments where qPCR detection is used, it should be considered the PCR can be affected by factors such as reaction efficiency (e.g. due to enzyme health), stoichiometry, and thermal cycling ramping which can lead to bias or artificial shift in results.

In embodiments where FISH detection is used, spores can be individually visualized. Further, in using FISH, filtering and concentrating samples of spores can lead to enhanced sensitive if an increase in sensitivity is desired or necessary. FISH can avoid biases, interference, and false positive and/or negative results that can arise using qPCR. FISH is a direct method for enumeration and visualization of spores without requiring cultivation and/or isolation of nucleic acids. FISH can also be used to determine the identity of spore.

DNA microarrays, terminal restriction fragment length polymorphism (TRFLP), denaturing gradient gel electrophoresis (DGGE), pyrosequencing, reverse transcription polymerase chain reaction (RT-PCR) can be suitable for determining the identity and/or diversity of spores, and cannot be used for direct enumeration and visualization. Q-PCR can be used for quantification of spores by utilizing an indirect method with the isolated nucleic acid.

Other nucleic acid detection techniques are identifiable by a skilled person upon reading the present disclosure. Other nucleic acid detection techniques are identifiable by a skilled person upon reading the present disclosure. Any detection technique which is based on detection of nucleic acids can be used in detecting spores which are permeabilized according to embodiments of the disclosure.

Some embodiments of the disclosure provide a method for detecting viable bacterial spores, in a sample.

The term “viable” as used herein with reference to microorganisms, and spores and in particular, to bacterial cells and endospores, refers to microbial entities having a metabolic rate compatible with life (e.g. bacterial vegetative cells) or an uncompromised, healthy spore coat (e.g. bacterial endospores). Exemplary viable bacteria comprise normal and intact bacterial cells and bacteria in a viable but nonculturable (VBNC) state, wherein the bacteria is in a state of very low metabolic activity and do not reproduce, but has the ability to become culturable once resuscitated (e.g. under a favorable growth condition). Exemplary viable endospores comprise endospores that have uncompromised, healthy spore coats and are capable of germinating under appropriate conditions; this includes those that exist in a VBNC state. Bacterial cells and endospores can enter the VBNC state in response to, for example, stress due to adverse nutrient, temperature, osmotic, oxygen, and light conditions.

The term “non-viable” as used herein with reference to microorganisms and spores, and in particular, to bacterial cells and endospores refers to microbial entities having a metabolic rate which is lower than a rate compatible with life (e.g. lower than a rate of vegetative cells) or having a compromised and/or damaged spore coat. Exemplary non-viable microorganisms do not have the ability to become culturable with attempted resuscitation (e.g., under a favorable growth condition). Exemplary non-viable endospores comprise spores that have compromised, damaged spore coats and are not capable of germinating. In particular, the coating of non-viable spore can be permeable to certain reagents of interest, including intercalating agents herein described

In some embodiments of the method directed to detect viable spores, permeabilization of the spores in the sample, is preceded by a treatment with an agent able to bind a nucleic acid of non-viable spores (herein also first form of nucleic acid) and render the first form of nucleic acid unavailable for detection performed following permeabilization. In particular, in those embodiments the agent can be an intercalating agent and the method comprises contacting the sample with an intercalating agent to intercalate and form a covalent linkage with the first form of nucleic acids.

An “intercalating agent” in the sense of the present disclosure indicates a chemical substance that can insert itself between base pairs in a nucleic acid molecule. The term “intercalating” as used herein indicates a process when a nucleic acid intercalating agent fits itself in between base pairs of a nucleic acid molecule. Structural and chemical properties that are characteristic of nucleic acid intercalating agents often comprise a proper molecular size, being cationic, polycyclic, aromatic, planar and hydrophobic for the following proposed mechanism of nucleic acid intercalation to take place: In aqueous isotonic solution, a cationic intercalating agent is attracted electrostatically to the polyanionic nucleic acid molecules. The intercalating agent displaces a sodium and/or magnesium cation that surrounds the nucleic acid, forming a weak electrostatic bond with the outer surface of the nucleic acid. From this position, the intercalating agent can slide into the hydrophobic environment found between the base pairs and away from the hydrophilic outer environment surround the nucleic acid. The base pairs transiently form such openings for the entering of the intercalating agent due to energy absorbed during collision with the solvent molecules. These structural modifications can lead to in vivo functional changes, such as inhibition of transcription, replication and/or repairing of the intercalated nucleic acid.

Intercalating agents of the disclosure can comprise_molecules having a nucleic acid intercalating moiety and a functional group capable of forming a covalent bond with a nucleic acid. In particular, intercalating agents of the disclosure further comprise a functional group capable of covalently binding to nucleic acids, for example, an azide.

The term “covalent binding” indicates a process of formation of a chemical bonding that is characterized by sharing of pairs of electrons between atoms, known as the covalent bond also referred to herein as a “covalent linkage”. Covalent bonding indicates a stable balance of attractive and repulsive forces between atoms when the atoms share their electrons, and includes many kinds of interaction, including σ-bonding, π-bonding, metal to metal bonding, agostic interactions, and three-center two-electron bonds. The term “electrostatic binding” indicates association between two oppositely charged entities.

Exemplary nucleic acid intercalating agents include propidium monoazide (PMA), ethidium monoazide bromide (EMA), ethidium bromide, berberine, proflavine, daunomycin, doxorubicin, and thalidomide

In particular, in some embodiments, an exemplary suitable intercalating agent is propidium monoazide (PMA) is shown below (Ref. 32, Ref. 33).

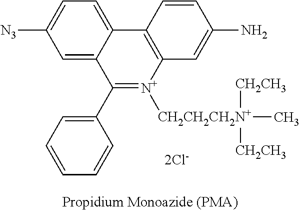

Other intercalating agents capable of selectively binding to accessible nucleic acids and further capable of photo-activated formation of a covalent bond with nucleic acid include but is not limited to ethidium monoazide bromide (EMA) shown below.

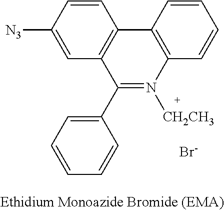

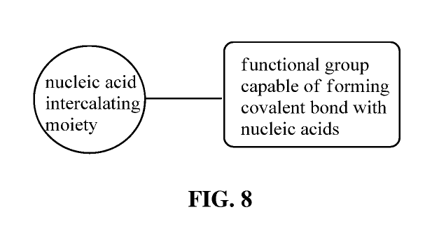

Further intercalating agents capable of selectively binding to accessible nucleic acids and capable of forming of a covalent bond with a nucleic acid are identifiable by a skilled person upon reading the present disclosure and can be any molecule comprising a nucleic acid intercalating moiety and a suitably located (e.g. in suitable proximity to react with nucleic acids after interaction) functional group capable of forming a covalent bond with a nucleic acid as shown in FIG. 8. For example, a planar molecule that facilitates a sliding in between and intercalating inter-nucleotide spaces of nucleic acids and comprising a functional group capable of forming of a covalent bond with a nucleic acid can be a suitable intercalating agent.

In embodiments of the methods and systems herein described, an intercalating agent used is able to bind to accessible nucleic acid in the sample which in some embodiments is typically provided by a nucleic acid of non-viable spore herein also first form of nucleic acid.

The term “nucleic acid” as used herein indicates a polymeric form of nucleotides of any length, either ribonucleotides or deoxyribonucleotides, that comprise purine and pyrimidine bases, or other natural, chemically or biochemically modified, non-natural, or derivatized nucleotide bases. Nucleic acids of the embodiments of the current disclosure include Deoxyribonucleic acid (DNA), ribonucleic acid (RNA), or DNA copies of RNA (complementary DNA or cDNA), which may be isolated from natural sources, recombinantly produced, or artificially synthesized. The nucleic acids may exist as single-stranded or double-stranded and any chemical modifications thereof, provided only that the modification does not interfere with amplification of selected nucleic acids. For example, the backbone of the nucleic acid can comprise sugars and phosphate groups or modified or substituted sugar or phosphate groups, and a nucleic acid may comprise modified nucleotides, such as methylated nucleotides and nucleotide analogs.

In particular, in embodiments herein described, the intercalating agent is able to intercalate and then covalently link nucleic acid of non-viable spores in view of structure of the coating that makes it permeable to the intercalating agent.

For example, PMA can access nucleic acids (DNA/RNA) of non-viable cells through physically ruptured and otherwise compromised membranes and once inside the cell it can intercalate with, and upon photo-activation with visible light, can form a covalent bond with nucleic acid (Ref. 33; Ref. 35). This can render the covalently-bound nucleic acids inaccessible for downstream molecular-based analytical techniques and other chemical manipulations.

In some embodiments, a sample of spores and in particular bacterial endospores can be treated with PMA for a suitable time (e.g. approximately 15 min) in “darkness” (e.g. a substantial absence of light). During this time, intercalation of the PMA with nucleic acids of the non-viable endospores can establish equilibrium. A concentration of an intercalating agent and amount of time for contacting the intercalating agent can vary depending on the particular intercalating agent. Determination of a suitable concentration can be performed by preliminary testing of PMA or other intercalating agent according to techniques identifiable by a skilled person upon reading of the present disclosure (see e.g. Example 10)

In some embodiments, following the intercalation, covalent linkage can be obtained with exposure of the sample to bright white light for approximately 5 minutes can be performed to photoactivate the PMA, and in particular the azide functionality to facilitate formation of a covalent bond with nucleic acids in the spore. Further techniques to provide covalent linkage of the intercalating agent are dependent on the specific agent and are identifiable by a skilled person. In some embodiments, treatments of a sample of spores with an intercalating agent capable of binding to nucleic acids of non-viable spores, followed by permeabilization of spores in a manner that is amenable to downstream analyses can allow, detection (e.g. a direct visualization and enumeration of spores) which can reduce and possibly minimize complications that accompany previously required germination regimes. A synergistic enzymatic and detergent weakening of the many spore layers as described herein can facilitate a structural compromising that can render the spores permeable without degrading the spore to a level, which precludes it from recognition.

In some embodiments, intercalation and subsequent formation of a covalent linkage with the nucleic acids can provide a first treated sample. The first treated sample can then be contacted with a protein degrading agent to allow permeabilization of bacterial spores to reagents for detection of a second form of nucleic acid comprised in bacterial spore not permeable to the intercalating agent. Permeabilization of the first treated sample can then provide a second treated sample. The second form of nucleic acid in the second treated sample is suitable for detection by nucleic acid detection techniques and provides an indicator of viable bacterial spores in the sample as the intercalated and covalently linked nucleic acids are not accessible for detection.

In these embodiments, detection techniques include, but are not limited to fluorescence in situ hybridization (FISH), Alexa-FISH, DNA microarrays, terminal restriction fragment length polymorphism (TRFLP), denaturing gradient gel electrophoresis (DGGE), pyrosequencing, reverse transcription polymerase chain reaction (RT-PCR), and quantitative (qPCR), fluorescence activated cell sorting (FACS), flow cytometry (flow-FISH)

Accordingly embodiments of the disclosure allow detection agents to contact and form a stable complex with accessible nucleic acid (e.g. by permeabilization of spores), together with substantially less to no recognition, contact and formation of a stable complex with inaccessible nucleic acid (e.g. intercalated and covalently linked nucleic acids) present. The term “substantially” as used herein indicates an extent of that does not materially affect the parameter at issue.

In some embodiments, the intercalating agent can selectively penetrate a compromised bio-membrane, such as cell membrane of dead bacteria and spore coat of dead endospores, but are substantially impermeable to intact bio-membrane of viable cells and healthy spore coats of endospores. After penetrating through the compromised spore coats of dead endospores, the agent is able to access and intercalate the nucleic acids.

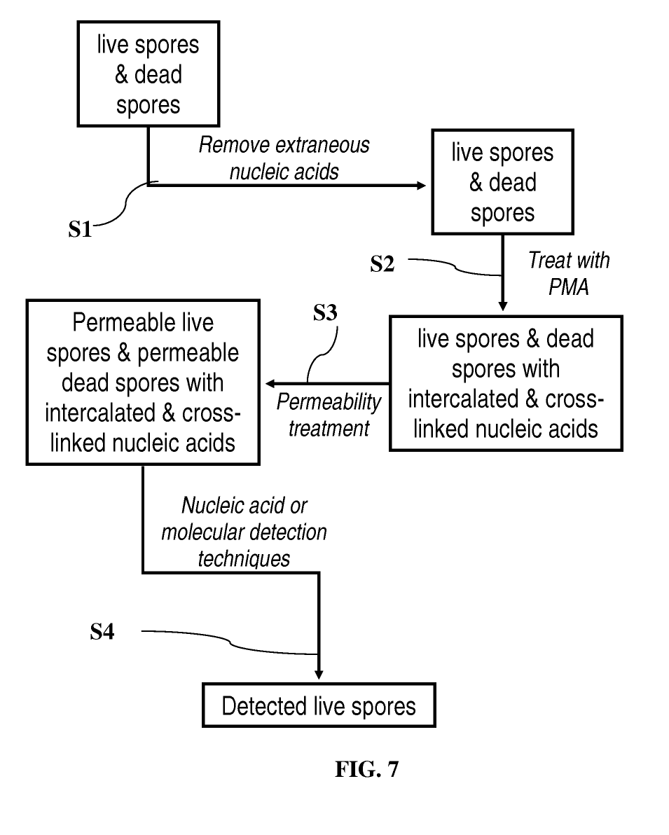

An exemplary method of the disclosure for detecting viable (live) bacterial spores is shown in FIG. 7. Starting with a sample comprising live and dead spores, the method comprises removing extraneous DNA from the sample followed by treatment with PMA (or other suitable agents comprising an intercalating moiety and a function group capable of forming a covalent bond to nucleic acids) and bright white light. At this point the PMA is only capable of accessing the DNA of endospores with compromised spore coats (i.e., dead spores), resulting in a sample comprising live spores plus dead spores, wherein the nucleic acids arising from the dead spores have been intercalated and cross-linked by the PMA (or other suitable agents comprising an intercalating moiety and a function group capable of forming a covalent bond to nucleic acids)—rendering these nucleic acids inaccessible to downstream FISH procedures. A permeability treatment, capable of permeating live and dead spores, then gives a sample comprising permeable live spores with accessible nucleic acids and permeable dead spores still having their nucleic acids intercalated and cross-linked by the PMA (or other suitable agents comprising an intercalating moiety and a function group capable of forming a covalent bond to nucleic acids) and thus remaining inaccessible. Numerous nucleic acids detection techniques can then be performed to detect the live spores as already described herein.

In some embodiments, a relative proportion of viable bacterial endospores (i.e., a live/dead assay) can be determined in a sample. For example, a given sample be split into equivalent fractions (portions/units) and each portion tested in parallel. A first unit of the sample can be tested according to several embodiments herein described, to serve as an indicator of a number of viable or non-viable spores. A second unit of the sample can be tested according to according to several embodiments herein described, to indicate a number viable and non-viable endospores (e.g. to provide a total number of endospores). A comparison of the number viable endospores to a total number of endospores or a comparison of a number non-viable endospores to a total number of endospores can provide a proportion of viable or non-viable endospores, respectively.

In several embodiments, methods for performing a live/dead assay for bacterial spores are provided. Such methods comprise splitting a sample of spores to be tested into portions/units and performing tests in parallel.

For example, the method can comprise contacting a first unit of the sample with an intercalating agent capable of binding a first form of nucleic acids comprised in bacterial spores permeable to the intercalating agent. The first form of nucleic acid comprises nucleic acids which are comprised in bacterial spores that are permeable to the intercalating agent.

In this embodiment, the intercalating agent is capable of emitting an intercalating labeling signal. Exemplary intercalating agents comprise PMA, EMA, and other agents capable of intercalating nucleic acids and capable of forming a covalent linkage with the nucleic acids as already described herein. In these embodiments, the contacting is performed for a time and under condition to allow intercalation of the intercalating agent in the first form of nucleic acid and covalent linkage of the intercalating agent with the first form of nucleic acid following intercalation, which results in a treated first unit.

A detection of the first form of nucleic acid in the treated first unit by detecting the intercalating labeling signal is an indicator of non-viable bacterial spores. The detection can be performed, for example, by subjecting the sample to fluorescent microscopy analysis.

In these embodiments, the method further comprises contacting a second unit of the sample with a protein degrading agent for a time and under condition to allow permeabilization of bacterial spores to reagents for detection of nucleic acid comprised in bacterial spores as previously described herein to obtain a second treated sample.

A detection of nucleic acids comprised in bacterial spores in the second treated sample (e.g. via FISH) is an indicator of viable and non-viable bacterial spores in the sample.

In these embodiments, the indication of non-viable spores together with the indication of viable and non-viable spores can provide a quantitative detection and in particular a relative proportion of viable and/or non-viable spores. For example, if the indication of non-viable spores is a quantitative indication of spores and the indication of viable and non-viable spores (e.g. from detection of the second treated sample) is also a quantitative indication, a number of non-viable spores (e.g. from the quantitative indication of non-viable spores from the detection of the first treated sample) can be compared to a number of total spores (e.g. from the quantitative indication of viable and non-viable spores from the detection of the second treated sample) to provide a proportion of non-viable spores. A proportion of viable spore can be obtained by subtracting the number of non-viable spores from a total number of spores.

In another embodiment of a live/dead assay, a first unit of the sample with an intercalating agent (e.g. PMA, EMA) capable of binding a first form of nucleic acids comprised in bacterial spores permeable to the intercalating agent. The first form of nucleic acid comprises nucleic acids which are comprised in bacterial spores that are penetrable by the intercalating agent (e.g. non-viable spores). In embodiments of the method, the contacting is performed for a time and under condition to allow intercalation of the intercalating agent in the first form of nucleic acid and covalent linkage of the intercalating agent with the first form of nucleic acid following intercalation according to embodiments herein described. In the method, the contacting results in a first treated unit that is further contacted with a protein degrading agent for a time and under condition to allow permeabilization of bacterial spores to reagents for detection of a second form of nucleic acid. The second form of nucleic acids is comprised in bacterial spores not permeable to the intercalating agent to obtain a secondly treated first unit. A detection (e.g. by FISH) of the second form of nucleic acid in the secondly treated first unit indicates viable bacterial spores in the first unit.

The method further comprises contacting a second unit of the sample with a protein degrading agent for a time and under condition to allow permeabilization of bacterial spores to reagents for detection of a nucleic acid comprised in bacterial spores permeable or not permeable to the intercalating agent to obtain a treated second unit according to embodiments herein described and detecting the nucleic acid in the treated second unit to indicate viable and non-viable bacterial spores in the second unit.

In this embodiment, the indication of viable spores together with the indication of viable and non-viable spores can provide a relative proportion of viable and/or non-viable spores. For example, if the indication of viable spores is a quantitative indication of spores and the indication of viable and non-viable spores (e.g. from detection of the second treated sample) is also a quantitative indication, a number of non-viable spores (e.g. from the quantitative indication of non-viable spores from the detection of the first treated sample) can be compared to a number of total spores (e.g. from the quantitative indication of viable and non-viable spores from the detection of the second treated sample) to provide a proportion of non-viable spores. A proportion of viable spore can be obtained by subtracting the number of non-viable spores from a total number of spores.

The phrase “indicator of” as used herein with reference to viable spores refers to quantitative or qualitative detection of the labeling signal emitted from a labeled molecule (such as the second form of a nucleic acid) associated with viable spores in a sample, such that there is a statistical significant and in particular a highly statistically significant probability that the labeling signal is indeed associated with the viable spores and not due to the undesired detection of non-viable spores in the sample (e.g. due to the non-viable spores being sufficiently and unexpectedly impermeable to the intercalator).

The phrase “indicator of” as used herein with reference to non-viable spores refers to quantitative or qualitative detection of the label signal emitted by a labeled molecule (such as the first form of a nucleic acid) associated with non-viable spores in a sample, such that there is a there is a statistical significant and in particular a highly statistically significant probability that the labeling signal is indeed associated with the non-viable spores and not due to the undesired detection of viable spores in the sample (e.g. due to the viable spores being sufficiently and unexpectedly permeable to the intercalator).

The term “labeled molecule” or “probe” as used herein as component of a complex or molecule refer to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like that are identifiable by a skilled person. As a consequence, the wording “signal” or “labeling signal” as used herein indicates the signal emitted from the agent itself or the probe conjugated to the agent that allows detection of the agent and consequently the labeled target, including but not limited to radioactivity, fluorescence, chemiluminescence, production of a compound in outcome of an enzymatic reaction and the like that are identifiable by a skilled person.

Further embodiments of the disclosure provide methods determining the abundance of viable bacterial endospores remaining after artificially encapsulating the bacterial endospores within a model spacecraft material, poly(methylmethacrylate) (PMMA; Lucite, Plexiglas), and releasing with an organic solvent (PolyGone-500; RPM technologies LLC, Reno, Nev.). PMMA polymers are commonplace in a multitude of industries, ranging from biotech and aerospace to pharmaceuticals and medicine.

Additional microorganisms rendered detectable through the methods of this disclosure, which have analogous layers, compositions, and/or coatings, will be understood by a skilled person.

In particular, in some embodiments the spores to be permeabilized and detected according to methods of the disclosure are bacterial spores and in particular endospores, in some embodiments the spores are those of other spore-forming microorganisms such as protozoa, as well as some plants, algae, and fungi.

In some embodiments, a permeabilization and/or detection of nucleic acids in oocysts and conidia can be performed.

An oocyst is cyst containing a zygote formed by a parasitic protozoan such as the malaria parasite. A conidia is an asexually produced non-motile fungal spore, formed on a conidiophores, examples of which include but are not limited to blastoconidia, arthroconidium, annelloconidium, phialoconidium, poroconidia and aleurioconidia. In particular, the term “conidia” refers to a spore produced in vegetative reproduction of a fungus and located on sporulating blotches. Fungi can produce two different types of conidia: the largest one are called macroconidia and the smallest, micoconidia. Conidia are produced and beared by specialized mycelial hyphae called conidiophores.

Oocysts and conidia are structurally similar to bacterial spores in that they also comprise a layer protecting nucleic acids in their core. Accordingly, in some embodiments, a permeabilization and detection of viable fungal conidia and viable protozoa oocysts can be performed according to previously described embodiments of the disclosure. In these embodiments, permeabilization conditions can be modified to be more suitable for permeabilization of the particular layers comprising the protective layer for oocysts and conidia.

In some embodiments methods and systems herein described allow detection of biocontamination, fabrication, assembly and processing of many industrial products, including medical devices, pharmaceuticals, spacecraft components, and bulk and canned foods are carried out inside a clean room facility. (Ref. 16) (Ref. 72)(Ref. 20).

In some embodiments, permeabilization of spores in a manner that is amenable to downstream analyses are herein described which can allow a direct visualization and enumeration of spores, thus circumventing complications that accompany previously required germination regimes. A synergistic enzymatic and detergent weakening of the many spore layers as described herein can facilitate a structural compromising that is configured to render the spores permeable without degrading the spore to a level, which precludes it from recognition.

In some embodiments, a protocol for permeabilization can be coupled to a downstream, specific means of visualizing (e.g. FISH) microbial cells and spores with a chemical pretreatment which precludes a portion that is not living (non-viable; due to a compromised nature of their spore coats) from detection, thus providing an ability to selectively visualize and enumerate the living spores present in a given sample, in a molecular biological fashion without the need for heavily-biased cultivation-based methodologies.

As described herein, agents capable of selectively accessing and subsequently binding to the nucleic acids of non-viable endospores; agents capable of permeabilizing endospores; and agents capable of selectively accessing and binding to the nucleic acids of permeabilized viable endospores for detection of viable endospores in a sample as herein described can be provided as a part of systems to perform a detection, including any of the detection methods described herein.

The systems can be provided in the form of a kit of parts. In a kit of parts, agents capable of binding to the nucleic acids of non-viable endospores; agents capable of decoating or partially decoating endospores; agents capable of binding to nucleic acids of permeabilized viable endospores; and other reagents to perform the detection of viable endospores can be comprised in the kit independently. Primers can be included in one or more compositions, and agents can be in a composition together with a suitable vehicle.

Additional components can include labeled molecules and in particular, labeled polynucleotides, labeled antibodies, labels, microfluidic chip, reference standards, and additional components identifiable by a skilled person upon reading of the present disclosure.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (e.g. wash buffers and the like).

Applications for the methods and systems provided herein include but are not limited to planetary protection policy-making and implementation, medical, pharmaceutical, aquaculture, wastewater and drinking water treatment facilities, food-processing industries and for improved assessment of the embedded/encapsulated bacterial endospores burden.

For example, a disclosed capability to selectively and directly visualize and enumerate the living, viable fraction of a given spore assemblage (against a background of expired and damaged spores) via a culture independent fluorescence-based in situ microscopic method can be employed to provide a reliable account of the viable spore abundance and diversity associated with spacecraft. In some instances, such as transport into space, physiologically inactive, but viable spores, capable of germinating and becoming active are significant in that they are the only life forms that are likely to survive and present a risk of propagation.

### EXAMPLES

The methods and systems disclosed herein are further illustrated in the following examples, which are provided by way of illustration and are not intended to be limiting.

In particular, the following examples illustrate exemplary assay for identifying spores and related methods and systems. In particular, the following examples illustrate methods and systems for permeabilization of endospores B. pumilus, and for detecting viable and/or viable/non-viable spores of B. pumilus in a sample. In particular, the following examples show method to permeabilize spores using protein detergents and to detect spore using PMA intercalating agent and FISH reagents. A person skilled in the art will appreciate the applicability and the necessary modifications to adapt the features described in detail in the present section, to additional spores, bacteria or other microorganisms, protein degradation agents, intercalating agents, detection reagents and reactions, samples, solutions, methods and systems according to embodiments of the present disclosure

### Example 1

### Bacterial Strain and Endospore Preparation;

Bacillus pumilus strain SAFR-032, isolated from the surface of an active spacecraft assembly clean room at the Jet Propulsion Laboratory, Pasadena, Calif., was used as the model organism in this study due to its characteristic resistance to extreme temperature, H₂O₂ exposure, and UV and gamma radiation (Ref 12; Ref 28; Ref 10; Ref 15). A suspension of purified B. pumilus SAFR-032 spores was prepared as described previously (Ref 15).

Briefly, a single colony of B. pumilus SAFR-032 grown overnight on tryptic soy agar (TSA; Becton Dickinson, Franklin Lakes, N.J.) at 32° C. was inoculated into liquid nutrient sporulation medium (NSM) (Ref 29) and incubated at 32° C. with shaking at 150 rpm for 3 to 5 days. Purification of spores was accomplished by repeated washing with salts, detergents, and nuclease-free water. Purified spores were rinsed in aqueous ethanol (50%) and heat-shocked at 80° C. for 15 min in order to any remaining vegetative cells. Finally, spore pellets were resuspended in nuclease-free water and the final suspension was stored in a sterile glass tube at 4° C. until further use.

### Example 2

### Endospore Encapsulation in PMMA

Ten ml of purified spore suspension (ca. 1×10⁷ CFU/ml) were mixed thoroughly with 100 g of molecular grade PMMA powder (Sigma, St. Louis, Mo.) using a sterile spatula and dried at room temperature (ca. 25° C.). Prior to encapsulation, the dried spore-laden PMMA powder (ca. 1×10⁶ CFU/g) was sifted through a 40 mesh screen to ensure a uniform spore-laden PMMA particle size.

One gram of sifted spore-laden PMMA powder was then mixed thoroughly with 1 ml of purified molecular grade liquid methylmethacrylate (MMA; Sigma, St. Louis, Mo.) in a 50 ml Falcon tube (Fisher Scientific). Liquid MMA was purified by mixing 100 ml MMA with 2% NaOH in a separatory funnel. After settling, the lower aqueous portion was discarded and the upper organic portion of purified MMA was used for the encapsulation. The suspension was then cured in a water bath at 50° C. for 60 min, followed by cooling at room temperature for 24 h. This heating and cooling process resulted in the formation of a hard and translucent pellet of encapsulated spores in PMMA. A negative control (PMMA without spores) was prepared in a similar fashion as above, without the addition of spores to the PMMA powder.

### Example 3

### Release of Endospores from PMMA Via Organic Solvent

The release of spores from the fully cured and hardened spore-laden PMMA pellet (ca. 2 g) was achieved by adding 2 ml of PolyGone™ 500 (RPM Technology, LLC; Reno, Nev.) to the pellet in a sterile Corning glass bottle. The bottle was incubated overnight at room temperature with shaking at 160 rpm. The resulting viscous suspension was centrifuged at 12,000×g for 60 min, followed by air drying of the pellet for 60 min. One ml of sterile nuclease-free water was then added to the pellet, and the tube was vigorously vortex mixed for 20 min. The resulting suspension was washed twice with phosphate-buffered saline (PBS), and stored at 4° C.

### Example 4

### Propidium Monoazide Treatment

Prior to treatment with PMA, suspensions of purified B. pumilus SAFR-032 spores (ca. 1×10⁶ CFU/ml) kept at 4° C. (presumed to be alive, viable) or heat-killed at 121° C. for 15 min (presumed to be dead, non-viable); the PolyGone-released spore suspension; and the corresponding negative control (PMMA without spores) suspension; were treated with Promega RNase-free DNase to degrade contaminant extraneous DNA as per manufacturer's instruction (Promega, Madison, Wis.). Afterwards, the spore suspensions were washed thrice with PBS to remove the residual DNase, followed by addition of 4 μl of 4 mM PMA (Biotium, Calif.) into a 496 μl DNase-treated spore suspension in a clear 1.5 ml microfuge tube. PMA-treated spores were incubated in the dark for 15 min at room temperature with shaking (165 rpm), followed by exposure to a 500 W halogen light for 5 min on ice. Spores were then washed twice with PBS and subjected to FISH analysis.

### Example 5

### Spore Permeabilization

The purified B. pumilus SAFR-032 spores kept at 4° C. (presumed to be alive, viable), heat-killed at 121° C. for 15 min (presumed to be dead, non-viable), the PolyGone-released spores and corresponding negative control (PMMA without spore) samples with and without PMA treatment were subjected to permeabilization procedures. Bacterial endospores were subjected to permeabilization procedures as described by Applicants in details in Ref 25 incorporated herein by reference in its entirety. Briefly, the spores were first treated with 1 ml protease solution [100 mM Tris-HCl, 0.5% sodium dodecylsulfate (SDS) and 1.5 U proteinase-K] for 10 min at 35° C.

Treated spores were washed twice with PBS and partially decoated in 0.5 ml solution containing 10 M urea, 0.07 M Tris, 0.14 M Dithiothreitol (DTT), 2 mM EDTA, 1% SDS and 1% Triton X-100 for 15 min at 60° C. with shaking (150 rpm). Spores were once again washed twice with PBS, followed by a glycosidase (7 mg/ml lysozyme and 7 U mutanolysin) treatment at 35° C. for 15 min. Afterwards, spores were washed twice with PBS and resuspended in 1 mL PBS solution containing 0.5% SDS, 1% Triton X-100 and 2 mM EDTA with shaking at 160 rpm for 30 min. Permeabilized spores were then washed twice with PBS and resuspended in 200 μl PBS.

### Example 6

### FISH Analysis

FISH hybridization reactions were carried out in standard 0.5 ml PCR tubes containing hybridization solution of 20 mM Tris, 0.01% SDS, 30% formamide, 0.9 M NaCl and 10 ng/μl of EUB338 (5′-GCT GCC TCC CGT AGG AGT-3′-SEQ ID NO: 1) or non-EUB338 (5′-CGA CGG AGG GCA TCC TCA-3′ SEQ ID NO: 2) oligonucleotide probe labeled with Alexa Fluor® 488 dye at the 5′ terminal phosphate. Non-EUB338 was used to assess the non-specific binding of the probe. The hybridization solution was pre-heated at 46° C. for 30 min, followed by the addition of an appropriate volume of permeabilized spores or negative control (PMMA without spores) samples with and without PMA treatment.

This mixture (final volume of 100 μl) was then incubated again at 46° C. for 2 h. The hybridization reaction was stopped by the addition of cold 100 mM Tris and 50 mM EDTA (pH 7.4) solution. This quelled reaction solution was filtered through a 0.22 μm black polycarbonate filter (Millipore, Billerica, Mass.) with gentle pressure, and rinsed three times with 0.22 μm-filtered nuclease-free water. The filter was mounted on a glass slide with Vectashield mounting media (Vector Laboratories, Burlingame, Calif.) and examined with a BX60 epifluorescence microscope (Olympus, Tokyo, Japan). Micrographs were taken at 1000× magnification with a charge-coupled device camera (Optronics, Goleta, Calif.), and at least 30 microscopic fields (ca. 1-200 fluorescent spores) were counted for each sample. The total viable spore count was estimated as described previously (Ref 41). All experiments were repeated three times and resulting data are expressed as mean±standard deviation (SD).

### Example 7

### Detection of B. pumilus Endospores using FISH Detection Following Permeabilization

B. pumilus SAFR-032 spores kept at 4° C. (presumed to be alive, viable), were prepared, as described in Example 1, heat-killed at 121° C. for 15 min (presumed to be dead, non-viable), artificially encapsulated in PMMA as described in Example 2. Standard FISH-based methods descried in Example 6 were performed following permeabilization performed as described in Example 5.

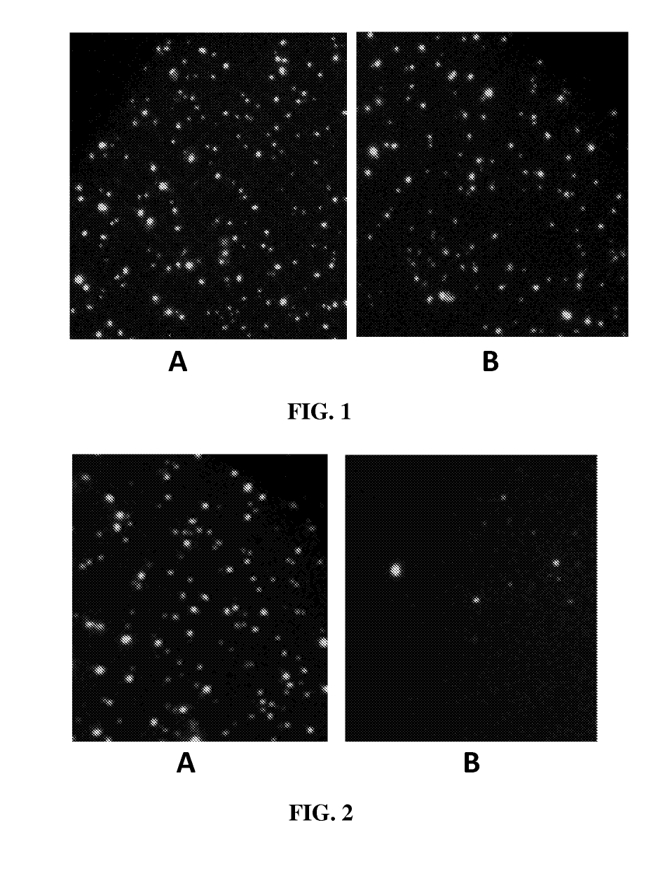

The results illustrated in FIG. 1 show that standard FISH-based methods without PMA pretreatment were able to detect a total of 0.652±0.074×10⁶ spores/ml (FIG. 1, Panel A) and 0.351±0.029×10⁵ spores/ml (FIG. 1, Panel B) of live and heat-killed spores, respectively.

In particular these results support the conclusion that permeabilization followed by traditional FISH techniques alone, although suitable to detect spores cannot effectively distinguish viable from non-viable spores. Previous studies have also documented the limitations of standard FISH techniques in determining the viability of bacterial cells (reviewed in Ref 7).

### Example 8

### Detection of B. pumilus Endospores Using PMA-FISH Treatment Following Permeabilization

B. pumilus SAFR-032 spores kept at 4° C. (presumed to be alive, viable), were prepared, as described in Example 1, heat-killed at 121° C. for 15 min (presumed to be dead, non-viable), artificially encapsulated in PMMA as described in Example 2. The spores were then pretreated with PMA a nucleic acids intercalating dye, as described in Example 4, prior to performing permeabilization with procedures described in Example 5 and FISH assay as described in Example 6.

In particular, preliminary experiments with live and heat-killed spores treated in a gradient from 4 to 48 μM PMA were performed. The results illustrated indicated ability of PMA-FISH analysis to distinguish viable and non-viable spores and that 32 μM was the most optimum PMA concentration for downstream FISH analyses. In these experiments, above 36 μM PMA, a strong inhibition was observed as total spore numbers resulting from FISH reactions dropped by ca. 60%.

FISH analysis with PMA-treated live and heat-killed spores yielded a spore abundance of 0.236±0.041×10⁶ (FIG. 2, Panel A) and 0.422±0.025×10³ (FIG. 2, Panel B) spores/ml, respectively. By comparison, the density of heat-killed spores from triplicate measurements was recorded as 0.078±0.003×10³ CFU/ml using standard spread plate methods on TSA

The results of the present study using a novel PMA-FISH method demonstrate that applying a PMA pretreatment to bacterial endospores prior to FISH analysis precludes the fraction of the spore population that is not living (non-viable) from detection. In this study, PMA-FISH was effectively able to discriminate live from non-viable spores, showing a 3 log lower detection of heat-killed spore populations compared to those of live spores, which was consistent with the results of time consuming standard cultivation based spread plate methods.

### Example 9

### Detection of B. pumilus Endospores Using PMA-FISH Treatment Following Permeabilization Under Different Reaction Conditions

B. pumilus SAFR-032 spores kept at 4° C. (presumed to be alive, viable), were prepared as described in Example 1, heat-killed at 121° C. for 15 min (presumed to be dead, non-viable), artificially encapsulated in PMMA as described in Example 2, and released via PolyGone-500 organic solvent as described in Example 3. The spores were pretreated with PMA, a nucleic acids intercalating dye, as described in Example 4, prior to performing permeabilization as described in Example 5 and FISH assay as described in Example 6.

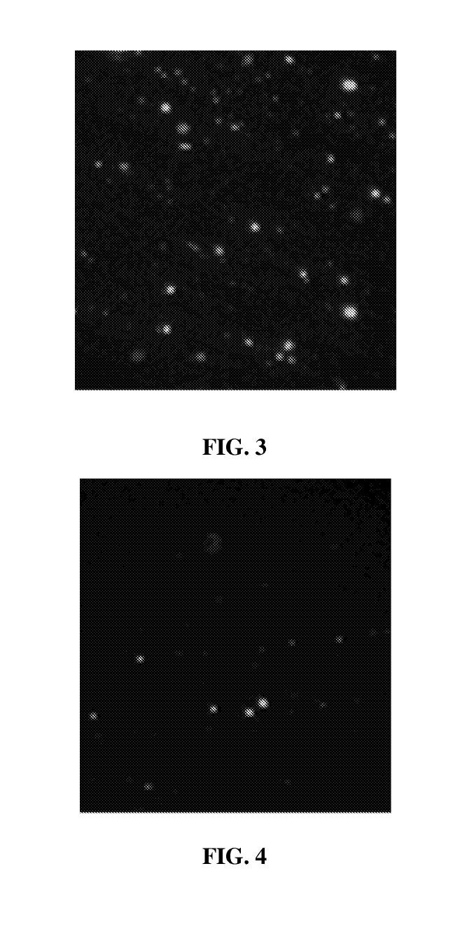

The results of the standard FISH analysis of the PMMA encapsulated spores released via PolyGone-500 so prepared are illustrated in FIG. 3 and FIG. 4.

In particular the results illustrated in FIG. 3 and FIG. 4 indicated a detectable spore density of 2.055±0.361×10⁵ spores/g (FIG. 3). However, PMA-FISH analyses of the very same PMMA-encapsulated spore stock yielded a spore density of 0.619±0.059×10³ spores/g (FIG. 4).

Enumeration of PMMA-encapsulated spores released via PolyGone-500 from triplicate measurements using standard spread plate methods on TSA yielded a density of 0.297±0.013×10³ CFU/g. Neither traditional FISH nor PMA-FISH methods using the non-EUB338 probe yielded any detectable fluorescence in live, heat-killed, or PMMA-encapsulated spores indicating that there was no appreciable non-specific binding of the Alexa Fluor® 488 dye. Negative control samples (PMMA without spores) assessed via traditional FISH and PMA-FISH methods using EUB338 and non-EUB338 probes did not yield any detectable fluorescence.

A 2 log reduction in the density of PMMA encapsulated spores released via PolyGone-500 was recorded by PMA-FISH, indicative of the deleterious effect of PolyGone-500 on the spore coat layers. The density of PMMA-encapsulated spores recorded via PMA-FISH was also consistent with the results of CFU obtained via spread plating on TSA.

### Example 10

### Identification of a Desirable PMA Concentration for Detecting B. Pumilus Endospores Using PMA-FISH Treatment Following Permeabilization

B. pumilus SAFR-032 spores kept at 4° C. (presumed to be alive, viable), were prepared, heat-killed at 121° C. for 15 min (presumed to be dead, non-viable), artificially encapsulated in PMMA and released via PolyGone-500 organic solvent as described in Examples 1 to 3. The spores were pretreated with PMA, a nucleic acids intercalating dye, prior to performing permeabilization and FISH assay as described in Examples 4 to 6.

In particular, the live and heat-killed spores were pretreated in a PMA gradient from 4 to 48 μM and the related effects on the. The results illustrated indicated that 32 μM was the most optimum PMA concentration for downstream FISH analyses. In these experiments, above 36 μM PMA, a strong inhibition was observed as total spore numbers resulting from FISH reactions dropped by ca. 60%.

### Example 11

### Methods to Confirm Viability of Endospores Detected Via Permeabilization/FISH or PMA-Permeabilization-FISH Detection

Various methods can be used on a sample to confirm viability determined using any one of the methods herein described. In addition to standard culture-based plate-counting (on TSA medium) methods, various biochemical and molecular methods, such as ATP bioluminescence (Ref 19), terbium dipicolinate fluorescence spectroscopy and microscopy (Ref 43), SYTO16-coupled flow cytometry and fluorescence microscopy (Ref 36; Ref 24), membrane potential dyes-coupled flow cytometry (Ref 17) and most-probable-number-rapid-viability (MPN-RV)-PCR (Ref 11; Ref 21) have been developed for the determination of viability of bacterial spores. The majority of these methods rely on the addition of germinants, such as amino acids, monosaccharides and/or dipicolinic acid (DPA) to the bacterial spore samples. In the presence of these germinants, the rationale is such that only spores capable of becoming metabolically active will germinate into vegetative cells.

Even if documented that such germination-dependent methodologies can underestimate viable spore numbers due to the transition to viable but non-culturable (VBNC) and/or active but-non-culturable (ABNC) physiological states in some species of bacteria (Ref 4) those methods can be effective if used in combination with any of the methods herein described.

Several fluorescent dyes, including acridine orange (Ref 14), 5-cyano-2,3-diotolyl tetrazolium chloride (CTC) (Ref 40), bis-(1,3-dibutylbarbituric acid) trimethine oxonol [DiBac₄(3)] (Ref 23), propidium iodide and SYTO9 (Ref 5) have been shown to foster rapid determination of viability of vegetative cells of bacteria. Even if variable results have been documented in efforts to determine endospore viability via the application of the fluorescent dyes due to the complex spore structure (Ref 18; Ref 22), those techniques can be effectively used in combination with any one of the methods herein described to confirm/determine viability of spores.

### Example 12

### Bacterial Strain and Spore Purification

Bacillus pumilus SAFR-032 strain used was isolated from the surfaces of an active spacecraft assembly facility clean room at the Jet Propulsion Laboratory, Pasadena, Calif., USA (Ref. 46, Ref. 47). B. pumilus SAFR-032 was grown overnight on tryptic soy agar (TSA; Becton Dickinson, Franklin Lakes, N.J., USA) at 32° C. and a single isolated colony was aseptically picked and transferred into liquid nutrient sporulation medium (NSM) (Ref. 50), which was incubated at 32° C. with shaking (150 rpm).

The following morning(s), wet mounts of the resulting culture were examined via phase contrast microscopy to determine the level of sporulation. Once the number of free spores in each culture exceeded 90% of the total number of entities present, typically after 3 to 5 days, cultures were subjected to spore purification. Spores were purified via repeated centrifugation (10,000×g at 4° C. for 10 min) and washing with various salts, detergents, and nuclease free water (Ref. 50). Purified spores were resuspended in 50% ethanol, heat-shocked (80° C. for 15 min) to ensure the inactivation of any remaining vegetative cells, and stored at 4° C. in sterile glass tubes.

### Example 13

### Encapsulation of Spores in PMMA

Analytical grade PMMA powder (100 g; Sigma, St Louis, Mo., USA) was seeded with 10 mL purified B. pumilus SAFR-032 spore suspension (described above; 1×10⁷ CFU/mL), which after drying and exhaustive mixing with a sterile spatula yielded a final spore density of approximately 1×10⁶ spores (CFU)/g. This spore-inoculated PMMA powder was then sifted through a 40-mesh screen to achieve uniform particles, and was stored in a sterile glass beaker.

To remove preservatives and impurities from the analytical grade liquid MMA (Sigma), 100 mL was mixed with 100 mL freshly prepared 2% NaOH in a 500 mL separatory funnel. The mixture was gently swirled, and upon separating into two fractions the lower fraction containing the preservatives was discarded. This washing procedure was repeated three times with 100 mL NaOH, followed by three additional rinses with nuclease-free water. The prepared MMA (approximately 90 mL) was then drained from the funnel, stored in a sterile glass bottle at 4° C., and was used within 24 hrs.

For each encapsulation event, 1 g prepared spore-laden PMMA powder was thoroughly mixed with 1 mL purified MMA in a 50 mL Falcon tube. The suspension was allowed to cure at 50° C. for 60 min and then cooled at room temperature for 24 hrs, resulting in the formation of a hard and translucent pellet. Negative control samples (PMMA+MMA) were prepared in an identical fashion without the addition of spores.

### Example 14

### Digestion of PMMA with Organic Solvent and Recovery of Spores

Spore-laden and negative control PMMA solids weighing approximately 2 g were placed in sterile Corning bottles, and 2 mL of either PolyGone™ 500 (RPM Technology, LLC; Reno, Nev., USA) or acetone was added. When the untreated spores were incubated for 24 hrs with organic solvent, a 1 log reduction in the spore density was recorded with a standard spread plate method (on TSA). To facilitate dissolving of the PMMA polymer, bottles were shaken at 160 rpm overnight at room temperature.

The resulting viscous suspension (approximately 4 mL) was centrifuged at 12,000×g for 60 min, at which time the supernatant was discarded and the pellet was air-dried for 60 min. Sterile nuclease-free water (1 mL) was added to the tube, and pellets were resuspended via thorough vortex mixing and repeated inversion for 20 min. The resulting spore suspension was transferred to a 1.5 mL microfuge tube, washed twice with PBS via centrifugation at 12,000×g for 15 min at 4° C., and was stored at 4° C.

### Example 15

### Spore Recovery Assessment Via DAPI and Epifluorescence Microscopy

The released spore suspensions and negative control (PMMA without spores) suspensions were subjected to DAPI (Invitrogen, Camarillo, Calif., USA) staining for visualization and enumeration via direct epifluorescence microscopy as per methods previously described by Porter and Feig (Ref. 51).

### Example 16

### Permeabilization of Spores

The test spore resuspension resulting from PMMA encapsulation and release was subjected to protease treatment in 1 mL solution containing 100 mM Tris-HCl, 0.5% sodium dodecylsulfate (SDS) and 1.5 U proteinase-K for 10 min at 35° C. The treated spores were washed twice with PBS via centrifugation at 12,000×g for 15 min, and partially decoated in 0.5 mL solution consisting of 10 M urea, 0.07 M Tris, 0.14 M dithiothreitol (DTT), 2 mM EDTA, 1% SDS and 1% Triton X-100 (or 0.5% Tween 80) for 15 min at 60° C. with shaking (150 rpm). Spores were harvested via centrifugation and washed twice with PBS (centrifugation at 12,000×g for 15 min), followed by glycosidase (7 mg/mL lysozyme and 7 U mutanolysin) treatment for 15 min at 35° C. The suspension was again washed twice with PBS (centrifugation at 12,000×g for 15 min) and resuspended in 1 mL PBS solution containing 0.5% SDS, 1% Triton X-100 (or 0.5% Tween 80) and 2 mM EDTA with shaking at 160 rpm for 30 min. The spores were washed twice with PBS (centrifugation at 12,000×g for 15 min) and finally resuspended in 200 μL PBS for use in Alexa-FISH microscopy. The permeabilization of never-encapsulated, purified B. pumilus SAFR-032 spores (termed positive control; approximately 10⁶ spores/mL) and negative control (PMMA without spores) samples was carried out using procedures identical to those described above for test spores.

### Example 17

### Alexa-FISH Oligonucleotide Probes

The universal probe EUB338 (5′-Alexa Fluor® 488-GCT GCC TCC CGT AGG AGT-3′) specific for eubacteria (Ref. 49) and a novel probe BP15 (5′-Alexa Fluor® 488-GGA TCA AAC TCT CCG AGG-3′) specifically designed for B. pumilus SAFR-032 were used in the Alexa-FISH experiments. The BP15 probe was designed using the computer program PRIMROSE (Ref. 52). Negative universal probe non-EUB338 (5′-Alexa Fluor® 488-CGA CGG AGG GCA TCC TCA-3′) and negative probe non-BP15 (5′-Alexa Fluor® 488-CCT CGG AGA GTT TGA TCC-3′) were also used to determine the level, if any, of non-specific binding of the probes. The specificity of each probe was tested with the Probe Match tool of the Ribosomal Database Project (rdp.cme.msu.edu/probematch/search.jsp). All probes were labeled with the Alexa Fluor® 488 dye at the 5′ terminal phosphate.

### Example 18

### Nucleic Acids Hybridization

All hybridizations described in the examples section were carried out in standard 0.5 mL PCR tubes containing 20 mM Tris, 0.01% SDS, 0.9 M NaCl and oligonucleotide probe at a concentration of 10 ng/μL in a final volume of 100 μL. In the case of the EUB338 probe, 30% formamide was added to the hybridization mixture to improve the hybridization efficiency. Hybridization solutions were incubated at 46° C. for 30 min, at which time an appropriate volume (variable depending on spore density) of permeabilized spore suspension was added and the reaction mixture was incubated at 46° C. for 2 hrs. Hybridization reactions were stopped by adding ice-cold 100 mMTris and 50 mMEDTA (pH 7.4), and subsequently filtered through 0.22 μm black polycarbonate filters (Millipore Inc., Billerica, Mass., USA) with gentle pressure. Filters were then rinsed three times with 0.22 μm filtered nuclease-free water. Filters were mounted on a glass slide with Vectashield mounting media (Vector Laboratories, Burlingame, Calif., USA) and examined with a BX60 epifluorescence microscope (Olympus, Tokyo, Japan) using a MWIB fluorescence cube (excitation 460-490 nm and emission 515-700 nm).

All micrographs were taken at a magnification of 1000× with an Optronics (Goleta, Calif., USA) charge-coupled device camera. At least 20 microscopic fields (approximately 20-200 fluorescent spores) were counted for each sample and the total count was determined as mentioned previously (Ref. 53). Hybridizations were also carried out on all samples with negative probes (non-BP15 or non-EUB338) on permeabilized positive control spores of B. pumilus SAFR-032 (those that had never been encapsulated), and on negative control (PMMA without spores) samples. All experiments were repeated three times and the resulting data were expressed as mean±standard deviation (SD).

### Example 19

### Detection of B. pumilus Endospores Using DAPI Dye and Alexa-FISH Treatment Following Permeabilization

B. pumilus SAFR-032 spores were prepared as described in Example 12, heat-killed at 121° C. for 15 min (presumed to be dead, non-viable), artificially encapsulated in PMMA as described in Example 13, and released via PolyGone-500 organic solvent as described in Example 14. The spores were recovered as described in Example 15, prior to performing permeabilization as described in Example 16 and FISH assay as described in Example 17.

The results of use of the commonly used nucleic acid-specific fluorescent DAPI dye to estimate the recovery of once-encapsulated spores released via PolyGone-500 or acetone, was a strong autofluorescence originating from simultaneous staining of PMMA- and solvent-associated organic debris precluded accurate enumeration. Additionally, the negative control (PMMA without spores) samples dissolved in PolyGone-500 or acetone also produced fluorescent signals due to uptake of DAPI dye by PMMA- and solvent-associated organic debris.

To circumvent this problem, an Alexa-FISH microscopy approach using novel BP15 (oligonucleotide specifically designed for B. pumilus SAFR-032 16S rRNA sequences) and EUB338 (oligonucleotide targeting all eubacterial 16S rRNA sequences) probes was evaluated for the enumeration of once-encapsulated recovered spores. FISH signals were not detected when spores (both non-treated and once-encapsulated) were pre-incubated for 24 hrs at 4° C. in a gradient of standard fixative reagents, including paraformaldehyde (1-10%, in 2% increments) and ethanol (20-70%, in 5% increments).

The complex and recalcitrant structure of bacterial endospores restricts the accessibility of fluorescent oligonucleotide probes for hybridization with nucleic acids at the spore core. To this end, various combinations of protease (proteinase-K), glycosidases (lysozyme and mutanolysin) and non-ionic surfactants (Tween 80 and Triton X-100) were tested to improve the permeabilization of control and test spores and thus achieve efficient FISH signaling for accurate enumeration. Table 1 summarizes the effects of treatment with each enzyme alone, and in combination with the surfactants, upon enumerating spores (untreated and those recovered from PMMA encapsulation and PolyGone-500 release) via Alexa-FISH microscopy with BP15 probes. The results demonstrated a synergistic action among the three enzymes and Triton X-100 that enabled the effective permeabilization of both positive-control and test spores for subsequent detection and enumeration by Alexa-FISH microscopy.

TABLE 1
No. spores detected after permeabilization using different enzymes
(1.5 U proteinase-K, 7 mg/mL lysozyme and 7 U mutanolysin)
and non-ionic surfactants (0.5% Tween 80 and 1% Triton X-100)†

| | Control spores | Encapsulated spores |
|---|---|---|
| Enzyme(s) | (×10⁵ spores/mL) | (×10⁵ spores/g) |
| No enzyme | 0 | 0 |
| Proteinase-K | 0.260 ± 0.024 | 0.177 ± 0.026 |
| Lysozyme | 0.590 ± 0.017 | 0.281 ± 0.038 |
| Mutanolysin | 0.200 ± 0.022 | 0.101 ± 0.013 |
| Proteinase-K + lysozyme + | 1.821 ± 0.253 | 0.778 ± 0.028 |
| mutanolysin | | |
| Proteinase-K + lysozyme + | 1.841 ± 0.261 | 0.780 ± 0.027 |
| mutanolysin + Tween 80 | | |
| Proteinase-K + lysozyme + | 6.590 ± 1.421 | 2.951 ± 0.383 |
| mutanolysin + Triton X-100 | | |

Each experiment was carried out in triplicate and results are expressed as mean ± SD.
†No. fluorescent spores was determined after hybridization with novel BP15 probe.
Encapsulated spores were recovered with PolyGone-500 solvent.

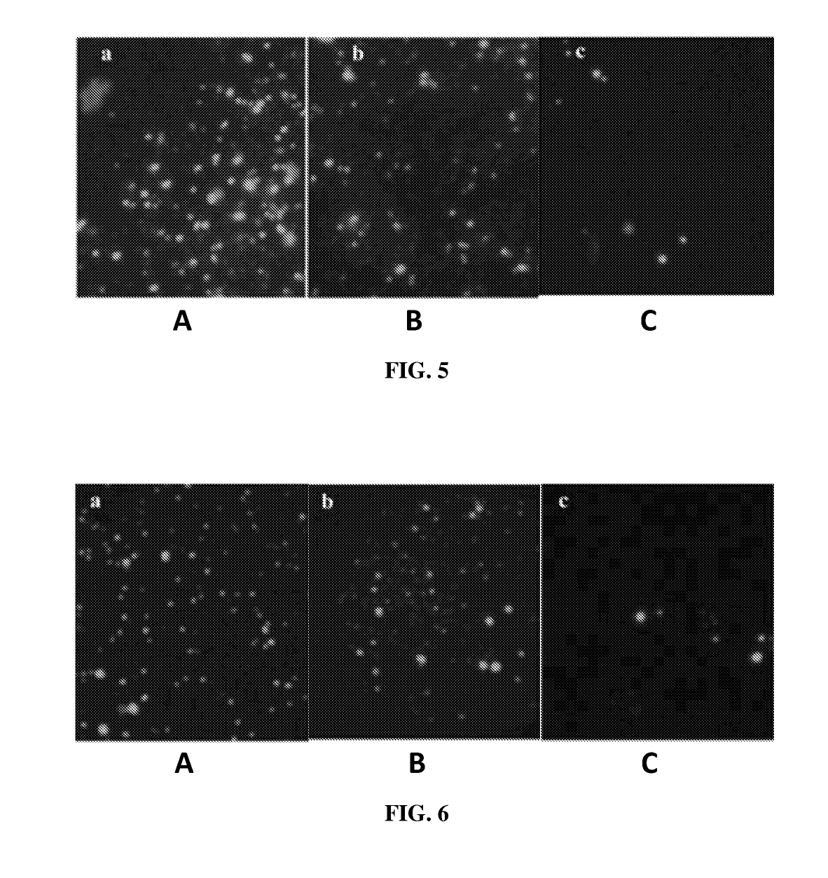

The novel BP15 probe used in concert with the novel permeabilization method referred to above was able to detect endospores in different viability conditions a as illustrated in FIG. 5. In particular, the illustration of FIG. 5 shows a total of 6.761±1.303×10⁵ non-treated control spores/mL (FIG. 5 Panel A), and 3.051±0.450×10⁵ spores/g (FIG. 5 Panel B) and 6.890±0.450×10⁴ spores/g (FIG. 5 Panel C) of PMMA-encapsulated spores extracted with PolyGone-500 or acetone, respectively.

The addition of 5% formamide to the hybridization reactions decreased (by approximately 95%) the detectable counts for both non-treated spores and PMMA-encapsulated spores, which indicated that formamide was not required for hybridization reactions with the novel BP15 probe. When the EUB338 universal probes were used in the absence of formamide the number of detectable untreated spores was 9.231±0.614×10³ spores/mL while counts of PMMA-encapsulated spores subsequently released via PolyGone-500 or acetone were 3.212±0.432×10³ spores/g and 9.540±1.061×10² spores/g, respectively.

### Example 20

### Identification of a Desirable Hybridization Conditions for Detecting B. PUMILUS Endospores Using Alexa-FISH Treatment Following Permeabilization

The reagents used in the experiments of Example 19 were tested using hybridization procedures described in Example 18.

In particular, a gradient of formamide (5-50%, in 5% increments) was tested to determine the stringency of and optimize the EUB338 hybridization reactions (Table 2). The detectable abundance of non-treated and encapsulated spores extracted with either PolyGone-500 or acetone increased by approximately 96% following the addition of 30% formamide, yielding counts of 6.262±1.405×10⁵ spores/mL (FIG. 6 Panel A), 2.150±0.370×10⁵ spores/g (FIG. 6 Panel B) and 6.603±0.316×10⁴ spores/g (FIG. 6 Panel C), respectively.

TABLE 2
Effect of formamide on the stringency of
hybridization of the EUB338 probe

| | | Encapsulated | Encapsulated- |
|---|---|---|---|
| Formamide | Control | PolyGone-500 | acetone |
| (%) | (×10⁵ spores/mL) | (×10⁵ spores/g) | (×10⁵ spores/g) |
| 0 | 0.092 ± 0.006 | 0.032 ± 0.004 | 0.009 ± 0.001 |
| 5 | 0.251 ± 0.032 | 0.108 ± 0.012 | 0.031 ± 0.003 |
| 10 | 0.622 ± 0.067 | 0.258 ± 0.026 | 0.085 ± 0.009 |
| 15 | 2.814 ± 0.173 | 0.281 ± 0.038 | 0.343 ± 0.033 |
| 20 | 5.751 ± 0.396 | 1.097 ± 0.112 | 0.594 ± 0.058 |
| 25 | 5.942 ± 0.638 | 2.064 ± 0.138 | 0.640 ± 0.065 |
| 30 | 6.262 ± 1.405 | 2.150 ± 0.370 | 0.660 ± 0.032 |
| 35 | 6.134 ± 0.823 | 2.085 ± 0.173 | 0.639 ± 0.064 |
| 40 | 4.382 ± 0.671 | 1.763 ± 0.176 | 0.533 ± 0.052 |
| 45 | 3.441 ± 0.413 | 1.354 ± 0.135 | 0.423 ± 0.042 |
| 50 | 2.810 ± 0.232 | 1.011 ± 0.106 | 0.318 ± 0.030 |

Each experiment was carried out in triplicate and results are expressed as mean ± SD.

Unlike with DAPI staining, the negative control (PMMA without spores) samples dissolved in PolyGone-500 or acetone did not stain to yield considerable levels of background fluorescent signal with neither the BP15 nor EUB338 probes. Furthermore, there were no fluorescent signals resulting from any of the permeabilized non-treated control spores, encapsulated spores released via PolyGone-500 or with acetone, and negative control (PMMA without spores) samples dissolved in PolyGone-500 or acetone hybridization reactions upon using the antisense non-BP15 and non-EUB338 probes. This confirmed the absence of non-specific binding of the Alexa Fluor® 488 dye.

An important aspect of the permeabilization protocol for B. pumilus SAFR-032 used the present example was the mixing and matching of various chemicals (EDTA, DTT, urea and SDS) and enzymes (protease and glycosidases) reported previously (Ref. 66, Ref. 67), and inclusion of additional Triton X-100 treatment steps. Triton X-100 is one of the most widely used non-ionic surfactants for lysing or permeabilizing prokaryotic and eukaryotic cell membranes (Ref. 68, Ref. 69). The efficacy of proteinase-K, lysozyme, mutanolysin and Triton X-100 were synergistic once thoroughly optimized with the BP15 probe, resulting in the detection of approximately 72% more spores than any protocol based on the combination of three enzymes only.

Neither of the Alexa-FISH probes was capable of detecting the true, 100% spore density (approximately 10⁶ spores/g) initially encapsulated in the PMMA. This might be attributed to the disintegration of spore borne genetic materials during the chemically abrasive encapsulation procedures, and/or the deleterious action of the organic solvents (PolyGone-500 or acetone). The novel BP15 and universal EUB338 Alexa-FISH probes used in this study did not yield detectable autofluorescence upon epifluorescent microscopic enumeration of encapsulated spores released with either PolyGone-500 or acetone. However, the signals emanating from hybridization events between Alexa-FISH probes and encapsulated spores varied dramatically between the two types of solvent tested to dissolve the model polymeric material (PMMA). When PolyGone-500 was used to dissolve the spore-laden PMMA solid, the percent recovery of detectable spores was 31% and 21% with BP15 and EUB338 probes, respectively. By comparison, these detectable recovery rates were only 6.9% with the BP15 probe and 6.6% with the EUB338 probe upon degrading the spore-laden PMMA solid with acetone. The exact mechanism causing this sharp decrease in Alexa-FISH signals in the presence of acetone was not examined. However, a possible explanation that is not intended to be limiting is that acetone might impose a more destructive impact on the spore.

### Example 21

### Additional Techniques for Confirming Detection of B. pumilus Endospores

Additional methods can be used to confirm detection of spores according to methods herein described and in particular exemplified.

Standard culture-based plate-counting (on TSA medium) methods are typically used for routine surveys of the presence and abundance of bacterial spores, a physiologically dormant form capable of withstanding extended periods of time in harsh conditions (e.g. extreme temperature, UV and γ radiation) (Ref. 44, Ref. 46, Ref. 48). In recent years, however, more attractive methods, such as microfluidic chips (Ref. 54), terbium dipicolinate fluorescence spectroscopy and microscopy (Ref. 55), most probable number (MPN)—PCR (Ref. 56), q-PCR (Ref. 57), ATP bioluminescence (Ref. 58) and FISH (Ref. 59) have been developed for more rapid and sensitive detection and identification of bacterial endospores. The majority of these approaches have focused primarily on enumerating spores by way of a coupling germinant, such as alanine and/or glucose, which initiates and drives the outgrowth and germination into vegetative cells (Ref. 45). It is well documented that such germination-dependent methodologies can underestimate spore numbers due to the transition to viable but not-cultivable (VBNC) physiological states (Ref. 60).

Direct epifluorescence microscopy techniques using DAPI dye have proven successful in enumerating spores originating from ice cores (Ref. 55), pure bacterial cultures (Ref. 61) and soil (Ref. 62). In the present study, DAPI staining procedures failed to prove effective due to the constant emission of background autofluorescence upon visualization of encapsulated spores released with either PolyGone-500 or acetone. Such autofluorescence could arise from the simultaneous staining of the composition of PMMA, and/or chemical deposits indigenous to the solvents used. The seemingly inescapable autofluorescence associated with DAPI-based microscopy necessitated the application of more sensitive FISH methodologies.

Preliminary testing with the BP15 and EUB338 probes on paraformaldehyde and ethanol treated control and encapsulated B. pumilus SAFR-032 spores demonstrated that Alexa-FISH tailored methods commonly used for vegetative cells were not effective for bacterial spores. Standard reagents (e.g. paraformaldehyde and ethanol) routinely used to treat vegetative cells for FISH analysis target relatively malleable phospholipid and lipopolysaccharide membranes (Ref. 63) whereas the structure of bacterial endospores is highly complex and rigid (Ref. 45, Ref. 64). Spores are surrounded by an exosporium composed mostly of proteinaceous compounds, followed by coat and cortex layers primarily composed of peptidoglycan and lipids (Ref. 45, Ref. 65).

### Example 22

### Detection of all (Viable and Non-Viable) Spores in a Sample

In this example, B. pumilus spores were permeabilized as indicated in Example 5 (without PMA treatment) and detected using the FISH analysis and fluorescence microscopy of Example 6. In this example, the permeabilization/detection was able to detect a total of 0.652±0.074×10⁶ spores/ml (FIG. 1, Panel A) and 0.351±0.029×10⁵ spores/ml (FIG. 1, Panel B) of live (prepared as in example 1) and heat-killed spores (prepared as in Example 1 and the heat killed at 120° C. for 15 min), respectively. This demonstrates an example of this particular method (the one of claim 4) to detect all bacterial spores.

### Example 23

### Detection of Viable Spores in a Sample

In this example, B. pumilus spores were prepared as in Example 1. A sample of these spores were left as is (representing viable spores) and another sample of these spores was heat killed at 120° C. for 15 min (representing non-viable spores). Each one of these samples was treated with the intercalating agent PMA as described in Example 4 so that the PMA was bound only to the nucleic acids of the non-viable spores. These two samples of PMA-treated spores (viable and non-viable) were then permeabilized as described in Example 5, and then subjected to detection by FISH analysis and fluorescence microscopy as in Example 6 to detect only the viable spores. In this example, the analysis yielded a spore abundance of 0.236±0.041×10⁶ (FIG. 2, Panel A) and 0.422±0.025×10³ (FIG. 2, Panel B) spores/ml, respectively, showing that viable bacterial spores could be differentially detected. Though the example uses separate samples of viable and non-viable bacterial spores, a skilled person could envision that the technique of this example can be applied to the analysis of a mixture of viable and non-viable spores to serve as an indicator of viable bacterial spores.

### Example 24

### Splitting of a Sample into Two Units and Detecting Only Non-Viable Spores in the First Unit and all Spores (Viable and Non-Viable) in the Second Unit

A skilled person would be able subject a first unit of a sample comprising viable and non-viable bacterial spores to the PMA treatment of Example 4 to cause PMA to bind only to the nucleic acids of the non-viable spores. A skilled person could then apply detection methods (such as fluorescence microscopy) specific for the detection of the labeling signal of the PMA thus detecting only the non-viable spores. A skilled person would also be able to subject a second unit of the sample to the conditions of, or similar to, Example 14 (permeabilization followed by detection) to detect all the bacterial spores in the second unit.

### Example 25

### Splitting of a Sample into Two Units and Detecting Only Viable Spores in the First Unit and all Spores (Viable and Non-Viable) in the Second Unit

In this example, a skilled person would be able to subject a first unit of a sample comprising viable and non-viable bacterial spores to the conditions of, or similar to, Example 16 (intercalator treatment, followed by permeabilization, followed by detection) to detect viable bacterial spores in the first unit. The skilled person would also be able to subject a second unit of the sample to the conditions of, or similar to, Example 14 (permeabilization followed by detection) to detect all the bacterial spores in the second unit.

The examples set forth above are provided to give those of ordinary skill in the art a complete disclosure and description of how to make and use the embodiments of the compositions, arrangements, devices, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### LIST OF REFERENCES

1. Amann R, and Fuchs B M. (2008) Single-cell identification in microbial communities by improved fluorescence in situ hybridization techniques. Nat Rev Microbiol, 6, 339-348.
2. Amann R I, Ludwig W, and Schleifer K H. (1995) Phylogenetic identification and in situ detection of individual microbial cells without cultivation. Microbiol Rev, 59, 143-169.
3. Bae S, and Wuertz S. (2009) Rapid decay of host-specific fecal Bacteroidales cells in seawater as measured by quantitative PCR with propidium monoazide. Water Res, 43, 4850-4859.
4. Barer M R, and Harwood C R. (1999) Bacterial viability and culturability. Adv Microb Physiol 41, 93-137.
5. Boulos L, Prevost M, Barbeau B, Coallier J, and Desjardins, R. (1999) LIVE/DEAD BacLight: application of a new rapid staining method for direct enumeration of viable and total bacteria in drinking water. J Microbiol Methods, 37, 77-86.
6. Brescia C C, Griffin S M, Ware M W, Varughese E A, Egorov A I, and Villegas, E N. (2009) Cryptosporidium propidium monoazide-PCR, a molecular biology-based technique for genotyping of viable Cryptosporidium oocysts. Appl Environ Microbiol, 75, 6856-6863.
7. Cenciarini-Borde, C, Courtois S, and La Scola, B. (2009) Nucleic acids as viability markers for bacteria detection using molecular tools. Future Microbiol, 4, 45-65.
8. COSPAR Planetary Protection Guidelines (2008) retrievable at the http:// website cosparhq.cnes.fr/Scistr/PPPolicy %2820-July-08%29.pdf.
9. Flekna G, Stefanic P, Wagner M, et al. (2007) Insufficient differentiation of live and dead Campylobacter jejuni and Listeria monocytogenes cells by ethidium monoazide (EMA) compromises EMA/real-time PCR. Res Microbiol, 158, 405-412.
10. Gioia J, Yerrapragada S, Qin X, Xiang H, Igboeli, O C, Muzny D, et al. (2007) Paradoxical DNA repair and peroxide resistance gene conservation in Bacillus pumilus SAFR-032. PLoS ONE 2, e928.
11. Kane S R, Letant S E, Murphy G A, Alfaro T M, Krauter P W, Mahnke R, Legler T C, and Raber E. (2009) Rapid, high-throughput, culture-based PCR methods to analyze samples for viable spores of Bacillus anthracis and its surrogates. J Microbiol Methods, 76, 278-284.
12. Kempf M J, Chen F, Kern R, and Venkateswaran K. (2005) Recurrent isolation of hydrogen peroxide-resistant spores of Bacillus pumilus from a spacecraft assembly facility. Astrobiology, 5, 391-405.
13. Kobayasi H, Oethinger M, Tuohy M J, Hall G S and Bauer T W. (2009) Improving clinical significance of PCR: use of propidium monoazide to distinguish viable from dead Staphylococcus aureus and Staphylococcus epidermidis. J Orthop Res, 27, 1243-1247.
14. Kogure K, Simidu U, and Taga N. (1979) A tentative direct microscopic method for counting living marine bacteria. Can J Microbiol, 25, 415-420.
15. La Duc M T, Dekas A, Osman S, Moissl C, Newcombe D, and Venkateswaran K. (2007) Isolation and characterization of bacteria capable of tolerating the extreme conditions of clean room environments. Appl Environ Microbiol, 73, 2600-2611.
16. La Duc M T, Kern R, and Venkateswaran K. (2004) Microbial monitoring of spacecraft and associated environments. Microb Ecol, 47, 150-158.
17. Laflamme C, Ho J, Veillette M, deLatremoille M C, Verrault D, Meriaux A, and Duchaine C. (2005) Flow cytometry analysis of germinating Bacillus spores, using membrane potential dye. Arch Microbiol, 183, 107-112.
18. Laflamme C, Lavigne S, Ho J, and Duchaine C. (2004) Assessment of bacterial endospore viability with fluorescent dyes. J Appl Microbiol, 96, 684-692.
19. Sakakibara, T., S. Murakami, N. Eisaki, M. Nakajima, and K. Imai. 1999. An enzymatic cycling method using pyruvate orthophosphate dikinase and firefly luciferase for the simultaneous determination of ATP and AMP (RNA). Analytical biochemistry 268:94-101.
20. Pacheco, F. L. C., and Pinto, T. D. A., 2010. The bacterial diversity of pharmaceutical clean rooms analyzed by the fatty acid methyl ester technique. PDA J. Pharm. Sci. Technol. 64, 156-166
21. Letant S E, Kane S R, Murphy G A, Alfaro T M, Hodges L R, Rose L J, and Raber E. (2010) Most-probable-number rapid viability PCR method to detect viable spores of Bacillus anthracis in swab samples. J Microbiol Methods, 81, 200-202.
22. Magge A, Setlow B, Cowan A E, and Setlow P. (2009) Analysis of dye binding by and membrane potential in spores of Bacillus species. J Appl Microbiol, 106, 814-824.
23. Mason D J, Allman R, and Lloyd D. (1993) Uses of membrane potential dyes with bacteria. In: Flow cytometry in microbiology. edited by D Lloyd, Springer-Verlag, London, United Kingdom, pp. 67-81.
24. Mathys A, Chapman B, Bull M, Heinz V, and Knorr D. (2007) Flow cytometric assessment of Bacillus spore response to high pressure and heat. Innovative Food Sci Emerging Technol, 8, 519-527.
25. Mohapatra B R, and La Duc M T (2012) Evaluation of fluorescence in situ hybridization to detect encapsulated Bacillus pumilus SAFR-032 spores released from poly(methylmethacrylate). Microbiol. Immunol. 56, 40-47.
26. Moter A, and Gobel U B. (2000) Fluorescence in situ hybridization (FISH) for direct visualization of microorganisms. J Microbiol Methods, 41, 85-112.
27. NASA. (1980) NASA Standard Procedures for the Microbiological Examination of Space Hardware, NHB 5340.1B, 1980. Jet Propulsion Laboratory Communication, National Aeronautics and Space Administration.
28. Newcombe D A, Schuerger A C, Benardini J N, Dickinson D, Tanner R, and Venkateswaran K. (2005) Survival of spacecraft-associated microorganisms under simulated martian UV irradiation. Appl Environ Microbiol, 71, 8147-8156.
29. Nicholson W, and Setlow P. (1990) Sporulation, germination and outgrowth. In: Molecular Biological Methods for Bacillus. edited by C R Harwood and S M Cuttings, John Wiley and Sons, Inc., Hoboken, N.J., p 391-450.
30. Nocker A, Richter-Heitmann T, Montijn R, Schuren F, and Kort R. (2010) Discrimination between live and dead cells in bacterial communities from environmental samples analyzed by 454 pyrosequencing. Int Microbiol, 13, 59-65.
31. Nocker A, Mazza A, Masson L, Camper A K, and Brousseau, R. (2009) Selective detection of live bacteria combining propidium monoazide sample treatment with microarray technology. J Microbiol Methods, 76, 253-261.
32. Nocker A, Sossa-Fernandez P, Bun M D, and Camper A K. (2007) Use of propidium monoazide for live/dead distinction in microbial ecology. Appl Environ Microbiol, 73, 5111-5117.
33. Nocker A, Cheung C Y, and Camper A K. (2006) Comparison of propidium monoazide with ethidium monoazide for differentiation of live vs. dead bacteria by selective removal of DNA from dead cells. J Microbiol Methods, 67, 310-320.
34. Oliver, A K. (2007) Use of propidium monoazide for live/dead distinction in microbial ecology. Appl Environ Microbiol, 73, 5111-5117.
35. Parshionikar S, Laseke I, and Fout G S. (2010) Use of propidium monoazide in reverse transcriptase PCR to distinguish between infectious and noninfectious enteric viruses in water samples. Appl Environ Microbiol, 76, 4318-4326.
36. Ragkousi K, Cowan A E, Ross M A, and Setlow P. (2000) Analysis of nucleoid morphology during germination and outgrowth of spores of Bacillus species. J Bacteriol, 182, 5556-5562.
37. Rawsthorne H, Dock C N, and Jaykus L A. (2009) PCR-based method using propidium monoazide to distinguish viable from nonviable Bacillus subtilis spores. Appl Environ Microbiol, 75, 2936-2939.
38. Regan J M, Oldenburg P S, Park H D, Harrington G W, and Noguera D R. (2003) Simultaneous determination of bacterial viability and identity in biofilms using ethidium monoazide and fluorescent in situ hybridization. Water Sci Technol, 47, 123-128.
39. Rogers G B, Stressmann F A, Koller G, Daniels T, Carroll M P, and Bruce K D. (2008) Assessing the diagnostic importance of nonviable bacterial cells in respiratory infections. Diagn Microbiol Infect Dis, 62, 133-141.
40. Rodriguez G G, Phipps D, Ishiguro K, and Ridgway H F. (1992) Use of a fluorescent redox probe for direct visualization of actively respiring bacteria. Appl Environ Microbiol, 58, 1801-1808.
41. Sherr E B, Caron D A, and Sherr B F (1993) Staining of heterotrophic protists for visualization via epifluorescence microscopy. In: Handbook of Methods in Aquatic Microbial Ecology. Edited by P F Kemp, B F Shen, E B Shen and J J Cole, CRC Press, Boca Raton, Fla., pp. 213-227.
42. Vesper S, McKinstry C, Hartmann C, Neace M, Yoder S, and Vesper A. (2008) Quantifying fungal viability in air and water samples using quantitative PCR after treatment with propidium monoazide (PMA). J Microbiol Methods, 72, 180-184.
43. Yung P T, Shafaat H S, Connon S A, and Ponce A. (2007) Quantification of viable endospores from a Greenland ice core. FEMS Microbiol Ecol, 59, 300-306.
44. Nicholson W. L., Munakata N., Horneck G., Melosh H. J., Setlow P. (2000) Resistance of Bacillus endospores to extreme terrestrial and extraterrestrial environments. Microbiol Mol Biol Rev 64: 548-72.
45. Setlow P. (2003) Spore germination. Curr Opin Microbiol 6: 550-6.
46. La Duc M. T., Kern R., Venkateswaran K. (2004) Microbial monitoring of spacecraft and associated environments. Microb Ecol 47: 150-8.
47. Kempf M. J., Chen F., Kern R., Venkateswaran K. (2005) Recurrent isolation of hydrogen peroxide-resistant spores of Bacillus pumilus from a spacecraft assembly facility. Astrobiology 5: 391-405.
48. Newcombe D. A., Schwerger A. C., Bernardini J. N., Dickinson D., Tanner R., Venkateswaran K. (2005) Survival of spacecraftassociated microorganisms under simulated Martian UV irradiation. Appl Environ Microbiol 71: 8147-56.
49. Amann R. I., Ludwig W., Schleifer K. H. (1995) Phylogenetic identification and in situ detection of individual microbial cells without cultivation. Microbiol Rev 59: 143-69.
50. Nicholson W., Setlow P. (1990) Sporulation, germination and outgrowth. In:Harwood C. R., Cuttings S. M., eds. Molecular Biological Methods for Bacillus. Hoboken, N.J.: JohnWiley and Sons Inc., pp. 391-450.
51. Porter K., Feig Y. (1980) The use of DAPI for identifying and counting aquatic microflora. Limnol Oceanogr 25: 943-8.
52. Ashelford K. E., Weightman A. J., Fry J. C. (2002) PRIMROSE: a computer program for generating and estimating the phylogenetic range of 16S rRNA oligonucleotide probes and primers in conjunction with the RDP-II database. Nucleic Acids Res 30: 3481-9.
53. Sherr E. B., Caron D. A., Sherr B. F. (1993) Staining of heterotrophic protists for visualization via epifluorescence microscopy. In:Kemp P. F., Shen B. F., Sherr E. B., Cole J. J., eds. Handbook of Methods in Aquatic Microbial Ecology. Boca Raton, Fla.: CRC Press, pp. 213-27.
54. Inami H., Tsuge K., Matsuzawa M., Sasaki Y., Togashi S., Komano A., Seto Y. (2009) Semi-automated bacterial spore detection system with micro-fluidic chips for aerosol collection, spore treatment and ICAN DNA detection. Biosens Bioelectron 24: 3299-305.
55. Yung P. T., Shafaat H. S., Connon S. A., Ponce A. (2007). Quantification of viable endospores from a Greenland ice core. FEMS Microbiol Ecol 59: 300-6.
56. Letant S. E., Kane S. R., Murphy G. A., Alfaro T. M., Hodges L. R., Rose L. J., Raber E. (2010) Most-probable-number rapid viability PCR method to detect viable spores of Bacillus anthracis in swab samples. J Microbiol Methods 81: 200-2.
57. Ryu C., Lee K., Yoo C., Seong W. K., Oh H. B. (2003) Sensitive and rapid quantitative detection of anthrax spores isolated from soil samples by real-time PCR. Microbiol Immunol 47: 693-9.
58. Lee J., Deininger R. A. (2004) A rapid screening method for the detection of viable spores in powder using bioluminescence. Luminescence 19: 209-11.
59. Laflamme C., Gendron L., Turgeon N., Filion G., Ho J., Duchaine C. (2009) Rapid detection of germinating Bacillus cereus cells using fluorescent in situ hybridization. J Rapid Meth Auto Microbiol 17: 80-102.
60. McDougald D., Rice S. A., Weichart D., Kjelleberg S. (1998) Nonculturability: adaptation or debilitation? FEMS Microbiol Ecol 25: 1-9.
61. Laflamme C., Lavigne S., Ho J., Duchaine C. (2004) Assessment of bacterial endospore viability with fluorescent dyes. J Appl Microbiol 96: 684-92.
62. Felske A., Akkermans A. D., De Vos W. M. (1998) In situ detection of an uncultured predominant bacillus in Dutch grassland soils. Appl Environ Microbiol 64: 4588-90.
63. Roller C., Wagner M., Amann R., Ludwig W., Schleifer K. H. (1994) In situ probing of gram positive bacteria with high DNA G+C content using 23S rRNA-targeted oligonucleotides. Microbiology 140: 2849-58.
64. Driks A. (1999) Bacillus subtilis spore coat. Microbiol Mol Biol Rev 63: 1-20.
65. Warth A. D. (1985) Mechanisms of heat resistance. In:Dring G. J., Ellar D. J., Goulds, G. W., eds. Fundamental and Applied Aspects of Bacterial Spores. London: Academic Press, pp. 209-25.
66. Filion G., Laflamme C., Turgeon N., Ho J., Duchaine C. (2009) Permeabilization and hybridization protocols for rapid detection of Bacillus spores using fluorescence in situ hybridization. J Microbiol Methods 77: 29-36.
67. Fischer K., Hahn D., Hönerlage W., Schönholzer F., Zeyer J. (1995) In situ detection of spores and vegetative cells of Bacillus megaterium in soil by whole cell hybridization. Syst Appl Microbiol 18: 265-73.
68. Helenius A., Simons K. (1975) Solubilization of membranes by detergents. Biochim Biophys Acta 415: 29-79.
69. Cimino M., Alamo L., Salazar L. (2006) Permeabilization of the mycobacterial envelope for protein cytolocalization studies by immunofluorescence microscopy. BMC Microbiol 6: 35.
70. Murray P., Baron E., Pfaller M., Jorgensen J., Yolken R. (2003) Manual of Clinical Microbiology, 8th edn., Vol. 1,Washington, D.C.: ASM Press.
71. Osman, S., Peeters, Z., La Duc, M. T., Mancinelli, R. Ehrenfreund, P., Venkateswaran, K. (2008). Effect of shadowing on survival of bacteria under conditions simulating the Martian atmosphere and UV radiation. Appl. Environ. Microbiol. 74, 959-970.
72. Nagarkar, P. P., S. D. Ravetkar, and M. G. Watve. 2001. Oligophilic bacteria as tools to monitor aseptic pharmaceutical production units. Appl. Environ. Microbiol. 67:1371-1374.

1. A method for permeabilizing a spore in a sample, the spore comprising a coating, the coating comprising a protein component, the method comprising:
contacting the sample with a protein degrading agent comprising a non-ionic detergent, the contacting performed for a time and under conditions to allow interaction of the protein degrading agent with the coating of the spore and degradation of the protein component.

2. The method of claim 1, wherein the protein degrading agent comprises a protein denaturing agent and an enzyme.

3. The method of claim 2, wherein the protein denaturing agent comprises a reducing agent and the enzyme is a protease.

4. The method of claim 1, wherein the protein degrading agent further comprises at least one of a metal chelator, a reducing agent, a glycosidase, and a buffer.

5. A system for permeabilizing a spore, the system comprising a non-ionic detergent and at least one additional protein degrading agent for simultaneous combined or sequential use in the method of claim 1.

6. A method for detecting spores, in a sample, the method comprising:
contacting the sample with a protein degrading agent for a time and under condition to allow permeabilization of spores to reagents for detection of a nucleic acid comprised in the spore, thus obtaining a treated sample, and
detecting the nucleic acid in the treated sample,
wherein the detected nucleic acid is an indicator of the spores in the sample.

7. The method of claim 6, wherein detecting the nucleic acid is performed a detection method selected from the group consisting of terminal restriction fragment length polymorphism (TRFLP), denaturing gradient gel electrophoresis (DGGE), pyrosequencing, reverse transcription polymerase chain reaction (RT-PCR), a DNA microarray analysis, quantitative (qPCR), fluorescence activated cell sorting (FACS), or flow cytometry (flow-FISH).

8. The method of claim 7, wherein detecting the nucleic acid is performed with FISH or Alexa-FISH.

9. A system for detecting spores in a sample, the system comprising a non-ionic detergent and reagents for detection of the nucleic acid for simultaneous combined or sequential use in the method of claim 6.

10. A method for detecting viable spores in a sample, the method comprising:
contacting the sample with an intercalating agent capable of binding a first form of nucleic acids comprised in spores permeable to the intercalating agent, the contacting performed for a time and under condition to allow intercalation of the intercalating agent with the first form of nucleic acid and covalent linkage of the intercalating agent with the first form of nucleic acid following intercalation, the contacting resulting in a first treated sample;
contacting the first treated sample with a protein degrading agent comprising a non-ionic detergent for a time and under condition to allow permeabilization of spores to reagents for detection of a second form of nucleic acid comprised in spore not permeable to the intercalating agent, thus obtaining a second treated sample; and
detecting the second form of nucleic acid in the second treated sample, the detected second form of nucleic acid being an indicator of viable spores in the sample.

11. The method of claim 10, wherein the intercalating agent is PMA.

12. The method of claim 11, wherein contacting the sample with an intercalating agent comprises performing a photo-activation of PMA to form the covalent linkage of the intercalating agent with the first form of nucleic acid of the non-viable spores.

13. The method of claim 10, wherein detecting the second form of the nucleic acid is performed with FISH or Alexa-FISH.

14. The method of claim 10, wherein detecting the second form of the nucleic acid is performed with a detection method selected from the group consisting of terminal restriction fragment length polymorphism (TRFLP), denaturing gradient gel electrophoresis (DGGE), pyrosequencing, reverse transcription polymerase chain reaction (RT-PCR), a DNA microarray analysis, quantitative (qPCR), fluorescence activated cell sorting (FACS), or flow cytometry (flow-FISH).

15. A system for detecting a viable spore, in a sample, the system comprising at least two of a non-ionic detergent, an intercalating agent and reagents for detection of the nucleic acid for simultaneous combined or sequential use in the method of claim 10.

16. A method for live/dead assay for spores in a sample, the method comprising
contacting a first unit of the sample with an intercalating agent capable of binding a first form of nucleic acids comprised in spores permeable to the intercalating agent, the intercalating agent capable of emitting an intercalating labeling signal, the contacting performed for a time and under condition to allow intercalation of the intercalating agent in the first form of nucleic acid and covalent linkage of the intercalating agent with the first form of nucleic acid following intercalation, the contacting resulting in a treated first unit;
detecting the first form of nucleic acid in the treated first unit by detecting the intercalating labeling signal, the detected first form of nucleic acid being an indicator of non viable spores;
contacting a second unit of the sample with a protein degrading agent comprising a non-ionic detergent for a time and under condition to allow permeabilization of bacterial spores to reagents for detection of nucleic acid comprised in spores thus obtaining a second treated sample; and
detecting the nucleic acid comprised in bacterial spores in the second treated sample, the detected nucleic acid being an indicator of viable and non-viable spores in the sample.

17. The method of claim 166, further comprising comparing the detected first form of nucleic acid in the first unit and the detected nucleic acid in the second unit to provide a live/dead proportion.

18. The method of claim 166, wherein the intercalating agent is PMA.

19. The method of claim 168, wherein contacting a first unit of the sample with an intercalating agent comprises performing a photo-activation of PMA to form the covalent linkage of the intercalating agent with the first form of nucleic acid of the non-viable spores.

20. The method of claim 166, wherein detecting the first form of the nucleic acid in the treated first unit is performed by fluorescence microscopy.

21. The method of claim 166, wherein detecting the nucleic acid comprised in spores in the second treated sample, is performed with FISH or Alexa-FISH.

22. The method of claim 166, wherein detecting the nucleic acid comprised in spores in the second treated sample is performed with a detection method selected from the group consisting of terminal restriction fragment length polymorphism (TRFLP), denaturing gradient gel electrophoresis (DGGE), pyrosequencing, reverse transcription polymerase chain reaction (RT-PCR), a DNA microarray analysis, quantitative (qPCR), fluorescence activated cell sorting (FACS), or flow cytometry (flow-FISH).

23. A method for a live/dead assay for spores in a sample, the method comprising
contacting a first unit of the sample with an intercalating agent capable of binding a first form of nucleic acids comprised in spores permeable to the intercalating agent, the contacting performed for a time and under condition to allow intercalation of the intercalating agent in the first form of nucleic acid and covalent linkage of the intercalating agent with the first form of nucleic acid following intercalation, the contacting resulting in a treated first unit;
contacting the treated first unit with a protein degrading agent comprising a non-ionic detergent for a time and under condition to allow permeabilization of spores to reagents for detection of a second form of nucleic acid comprised in spore not permeable to the intercalating agent, thus obtaining a secondly treated first unit;
detecting the second form of nucleic acid in the secondly treated first unit, the detected second form of nucleic acid being an indicator of viable spores in the first unit;
contacting a second unit of the sample with a protein degrading agent for a time and under condition to allow permeabilization of spores to reagents for detection of a nucleic acid comprised in spores permeable or not permeable to the intercalating agent, thus obtaining a treated second unit; and
detecting the nucleic acid in the treated second unit, the detected nucleic acid being an indicator of viable and non-viable spores in the second unit.

24. The method of 23, further comprising comparing the detected second form of nucleic acid in the first unit with the detected nucleic acid of the second unit to provide a live/dead proportion.

25. The method of claim 23, wherein the intercalating agent is PMA.

26. The method of claim 235, wherein contacting a first unit of the sample with an intercalating agent comprises performing a photo-activation of PMA to form the covalent linkage of the intercalating agent with the first form of nucleic acid of the non-viable spores.

27. The method of claim 23, wherein detecting the second form of nucleic acid in the secondly treated first unit is performed with FISH or Alexa-FISH.

28. The method of claim 23, wherein detecting the second form of nucleic acid in the secondly treated first unit is performed with a detection method selected from the group consisting of terminal restriction fragment length polymorphism (TRFLP), denaturing gradient gel electrophoresis (DGGE), pyrosequencing, reverse transcription polymerase chain reaction (RT-PCR), a DNA microarray analysis, quantitative (qPCR), fluorescence activated cell sorting (FACS), or flow cytometry (flow-FISH).

29. The method of claim 23, wherein detecting the nucleic acid in the treated second unit, is performed with FISH or Alexa-FISH.

30. The method of claim 23, wherein detecting the nucleic acid in the treated second unit, is performed with a detection method selected from the group consisting of terminal restriction fragment length polymorphism (TRFLP), denaturing gradient gel electrophoresis (DGGE), pyrosequencing, reverse transcription polymerase chain reaction (RT-PCR), a DNA microarray analysis, quantitative (qPCR), fluorescence activated cell sorting (FACS), or flow cytometry (flow-FISH).
